(19) United States
(12) Patent Application Publication
(10) Pub. No.: US 2015/0099667 A1
(43) Pub. Date: Apr. 9, 2015

(54) METHODS FOR THE ACTIVATION OF SILENT GENES IN A MICROORGANISM

(71) Applicant: Sanofi, Paris (FR)

(72) Inventors: Joachim Wink, Frankfurt am Main (DE); Stefan Bartoschek, Frankfurt am Main (DE); Andreas Batzer, Frankfurt am Main (DE); Stéphane Renard, Paris (FR)

(73) Assignee: Sanofi, Paris (FR)

(21) Appl. No.: 14/401,011

(22) PCT Filed: May 13, 2013
(86) PCT No.: PCT/EP2013/059811

§ 371 Date: Nov. 13, 2014

(30) Foreign Application Priority Data
May 14, 2012 (EP) 12305531.1

(51) Int. Cl. C12N 1/38; C12Q 1/02

(52) U.S. Cl. CPC C12N 1/38; C12Q 1/025; G01N 2500/10
USPC 506/10; 435/6.13; 435/244

(57) ABSTRACT

The present invention relates to a method for the activation of silent genes in microorganisms by co-cultivation of an inducer and a recipient microorganism. The inducer is selected from a chemical inducer, a microorganism inducer which is selected from a killed microorganism cell and/or inactivated culture medium in which said microorganism cell had been cultured and/or medium inducer. The present invention furthermore relates to a method for screening for an inducer and to a method of screening for a recipient microorganism by co-cultivation of an inducer and a recipient organism. The methods are useful for the detection of medicaments, such as antibiotics. The present invention further relates to media for culturing microorganisms comprising an inducer.

### FIELD OF THE INVENTION

The present invention relates to the activation of silent genes in microorganisms by co-cultivation of an inducer and a recipient microorganism. The present invention furthermore relates to a method for screening for an inducer and to a method of screening for a recipient microorganism by co-cultivation of an inducer and a recipient microorganism. The methods are useful in the detection of medicaments, such as antibiotics. The present invention further relates to media for culturing microorganisms comprising an inducer.

### BACKGROUND OF THE INVENTION

Natural products play a pivotal role in modern drug-based therapy of various diseases. Natural products from microorganisms are thereby a crucial source for novel drugs. It seems that many valuable drugs are overlooked when culturing microorganisms under standard laboratory conditions. This may be due to the fact that many biosynthesis genes remain silent and are activated only under specific conditions.

Activation of silent genes under chemical or physical stress conditions has been described in the art. One such condition for activating silent genes is by co-cultivation of different microorganisms. Co-cultivation may help to identify and develop new biotechnological substances. Watanabe et al. (1982) isolated a novel antibiotic producing bacterium by using the fungi Neurospora crassa, Aspergillus oryzae and Rhizopus hangchao as test organisms for co-cultivation. In addition to this, Meyer and Stahl (2003) reported that co-cultivation of Aspergillus giganteus with various microorganisms alters antifungal protein (afp) expression. The presence of Fusarium oxysporum triggered afp transcription, whereas dual cultures of Aspergillus giganteus and Aspergillus niger resulted in suppression of afp transcription. Schroeckh et al. (2009) showed that through individual co-cultivation of the fungus Aspergillus nidulans with a collection of 58 actinomycetes, silent fungal biosynthesis genes (not expressed under normal cultivation conditions) could be activated. They discovered that a direct interaction between the bacterial and fungal mycelia is required to activate the silent fungal biosynthesis genes. The review article of Bader et al. (2010) summarizes the findings in the art on microbial co-culture fermentations. In co-culture fermentations, interactions between different organisms play a critical role. Growth of cells may be enhanced or inhibited, or production of substances such as ethanol, hydrogen, lactic acid etc. may be increased. The review of Scherlach and Hertweck (2009) gives an overview on the strategies to trigger biosynthetic pathways to yield cryptic natural products. An et al. (2006) reported the co-cultivation of Pseudomonas aeruginosa and Agrobacterium tumefaciens to identify the molecular mechanisms that underlie multispecies microbial associations. It was found that Pseudomonas aeruginosa had a growth rate advantage over Agrobacterium tumefaciens. This reveals that quorum-sensing regulated functions and surface motility are important microbial competition factors for Pseudomonas aeruginosa.

The search for new drugs by microorganisms by means of classical cultivation methods has reached its limitations, which is seen by the sequencing of complete genomes. There are much more genes coding for secondary metabolites found in the genome than are expressed under standard conditions using standard media. Growth of bacteria under standard conditions using standard media leaves many proteins undetected and not available for characterisation of their potential applicability for pharmaceutical purposes. This disadvantages of the state of the art can be solved by using conditions for bacterial propagation that induce expression of silent genes coding for secondary metabolites. The therapeutic field for novel biological active secondary metabolites, especially antibiotics (e.g., against multi-resistant pathogenic bacteria), is still very important. Furthermore, there is a need for methods allowing the activation of silent genes that can be used in high throughput assays for the identification of new antibiotics or drugs. This problem is solved by the present invention.

### SUMMARY OF THE INVENTION

An embodiment of the invention provides a method for activation of silent genes in a recipient microorganism comprising co-cultivation of a recipient microorganism and an inducer that activates silent genes in the recipient microorganism, wherein the inducer is selected from the group consisting of a chemical inducer, a microorganism inducer, a killed microorganism cell, and inactivated culture medium in which the microorganism cell had been cultured. In a specific embodiment, the method is for screening for an inducer that activates silent genes in a recipient microorganism, the method comprising the steps of: (a) cultivating a recipient microorganism in the presence of a candidate inducer, and (b) determining the candidate inducer as being an inducer if silent genes are activated in the recipient microorganism, wherein the candidate inducer is selected from the group consisting of a candidate chemical inducer, a candidate microorganism inducer, a killed microorganism cell, and inactivated culture medium in which the microorganism cell had been cultured. In another specific embodiment, the method is for screening for a recipient microorganism, the method comprising the steps of: (a) cultivating a candidate recipient microorganism in the presence of an inducer that activates silent genes in the recipient microorganism, and (b) determining the candidate recipient microorganism as being a recipient microorganism if silent genes are activated in the candidate recipient microorganism, wherein the inducer is selected from the group consisting of a chemical inducer, a microorganism inducer, a killed microorganism cell, and inactivated culture medium in which the microorganism cell had been cultured.

In certain embodiments of the method, the activation of silent genes results in a change of a phenotype of the recipient microorganism, wherein the change of phenotype is a change of production of metabolites, a change of growth, and/or a change of morphology.

In certain embodiments of the method, the recipient microorganism is a microorganism selected from the group consisting of actinobacteria, myxobacteria, bacilli, and orfungi.

In certain embodiments of the method, the chemical inducer is selected from the group consisting of an anorganic salt of arsenic, plumb, cadmium, cobalt, selenium, nickel, strontium and/or nitride. In specific embodiments, the chemical inducer is AsI3, Pb(NO3)2, CdCl2, CoCl2, NaN3, NaHSeO3, NiCl2, and/or SrCl2, or DMSO.

In certain embodiments of the method, the microorganism inducer is a pathogenic microorganism or a soil microorganism selected from the group consisting of genus Acetobacter, Actinobacillus, Actinomadura, Actinomyces, Actinoplanes, Aeromonas, Alcaligenes, Alteromonas, Amycolatopsis, Arthrobacter, Aureobacterium, Bacillus, Bacteroides, Bifidobacterium, Borella, Brevibacterium, Burkholderia, Campylobacter, Cellulomonas, Clavibacter, Clostridium, Corynebacterium, Enterobacter, Enterococcus, Escherichia, Eubacterium, Flavobacterium, Fusobacterium, Haemophilus, Helicobacter, Klebsiella, Lactobacillus, Legionella, Microbacterium, Micrococcus, Micromonospora, Moraxella, Mycobacterium, Mycoplasma, Myxococcus, Neisseria, Nocardia, Pasteurella, Photorhabdus, Polyangium, Propionibacterium, Preoteus, Pseudomonas, Rhodococcus, Salmonella, Selenomonas, Serratia, Shigella, Sphingomonas, Staphylococcus, Streptococcus, Streptomyces, Thermoactinomyces, Treponema, Tsukamurella, Vibrio, Xanthomonas, Xenorhabdus or Yersinia. In other embodiments, the microorganism inducer is a pathogenic or soil fungusselected from the group consisting of Ascomycota, Basidiomycota, Oomycota, Zygomycota, yeasts, Escherichia coli ATCC 35218, Staphylococcus aureus ATCC 33592, Pseudomonas aeruginosa ATCC 27853, and Candida albicans ATCC 753.

In certain embodiments of the method, the method is a high-throughput method.

An embodiment of the invention provides a medium for cultivation of a recipient microorganism comprising an inducer that activates silent genes in the recipient microorganism, wherein the inducer is selected from the group consisting of a chemical inducer, a microorganism inducer, a killed microorganism cell, and inactivated culture medium in which the microorganism cell had been cultured.

In certain embodiments of the medium, the activation of silent genes results in a change of a phenotype of the recipient microorganism, wherein the change of phenotype is a change of production of metabolites, a change of growth, and/or a change of morphology.

In certain embodiments of the medium, the recipient microorganism is a microorganism selected from the group consisting of actinobacteria, myxobacteria, bacilli, and fungi.

In certain embodiments of the medium, the chemical inducer is selected from the group consisting of an anorganic salt of arsenic, plumb, cadmium, cobalt, selenium, nickel, strontium and/or nitride. In specific embodiments, the chemical inducer is AsI3, Pb(NO3)2, CdCl2, CoCl2, NaN3, NaHSeO3, NiCl2, and/or SrCl2, or DMSO.

In certain embodiments of the medium, the microorganism inducer is a pathogenic microorganism or a soil microorganism selected from the group consisting of genus Acetobacter, Actinobacillus, Actinomadura, Actinomyces, Actinoplanes, Aeromonas, Alcaligenes, Alteromonas, Amycolatopsis, Arthrobacter, Aureobacterium, Bacillus, Bacteroides, Bifidobacterium, Borella, Brevibacterium, Burkholderia, Campylobacter, Cellulomonas, Clavibacter, Clostridium, Corynebacterium, Enterobacter, Enterococcus, Escherichia, Eubacterium, Flavobacterium, Fusobacterium, Haemophilus, Helicobacter, Klebsiella, Lactobacillus, Legionella, Microbacterium, Micrococcus, Micromonospora, Moraxella, Mycobacterium, Mycoplasma, Myxococcus, Neisseria, Nocardia, Pasteurella, Photorhabdus, Polyangium, Propionibacterium, Preoteus, Pseudomonas, Rhodococcus, Salmonella, Selenomonas, Serratia, Shigella, Sphingomonas, Staphylococcus, Streptococcus, Streptomyces, Thermoactinomyces, Treponema, Tsukamurella, Vibrio, Xanthomonas, Xenorhabdus or Yersinia. In other embodiments, the microorganism inducer is a pathogenic or soil fungus selected from the group consisting of Ascomycota, Basidiomycota, Oomycota, Zygomycota, yeasts, Escherichia coli ATCC 35218, Staphylococcus aureus ATCC 33592, Pseudomonas aeruginosa ATCC 27853, and Candida albicans ATCC 753.

### BRIEF DESCRIPTION OF THE FIGURES

FIG. 1 is a bar chart showing percentage of extracts that showed >50% activities against one of the four assay strains (Escherichia coli ATCC 35218=EC, Staphylococcus aureus ATCC 33592=SA, Pseudomonas aeruginosa ATCC 27853=PA, and Candida albicans ATCC 753=CA).

FIG. 2 is a bar chart showing percentage of extracts that showed selective (>50%) activities against one of the four assay strains (Escherichia coli ATCC 35218=EC, Staphylococcus aureus ATCC 33592=SA, Pseudomonas aeruginosa ATCC 27853=PA, and Candida albicans ATCC 753=CA).

FIG. 3 is a bar chart showing percentage of different culture conditions of extracts, that showed >50% activity against the assay strain Escherichia coli ATCC 35218. MI=Actinobacteria strains that were cultivated in cultivation media where different microbial inducers were added, CI=Actinobacteria strains that were cultivated in cultivation media where different chemical inducers were added, STD=Actinobacteria strains that were cultivated in cultivation media.

FIG. 4 is a bar chart showing percentage of different microorganism/chemical inducers and cultivation media of the extracts that showed >50% activity against the assay strain Escherichia coli ATCC 35218. MI1=supernatant of Staphylococcus aureus ATCC 33592 cell culture, MI2=supernatant of Escherichia coli ATCC 35218 cell culture, MI3=supernatant of Pseudomonas aeruginosa ATCC 27853 cell culture, MI4=supernatant of Candida albicans ATCC 753 cell culture, MI5=cells of Staphylococcus aureus ATCC 33592, MI6=cells of Escherichia coli ATCC 35218, MI7=cells of Pseudomonas aeruginosa ATCC 27853, MI8=cells of Candida albicans ATCC 753, CI1=1.6 μg/ml AsI3, CI2=3.3 μg/ml AsI3, CI3=1.6 μg/ml Pb(NO3)2, CI4=3.3 μg/ml Pb(NO3)2, CI5=1.6 μg/ml CdCl2, CI6=3.3 μg/ml CdCl2, CI7=1.6 μg/ml CoCl2, CI8=3.3 μg/ml CoCl2, CI9=1.6 μg/ml NaN3, CI10=3.3 μg/ml NaN3, CI11=1.6 μg/ml NaHSeO3, CI12=3.3 μg/ml NaHSeO3, CI13=1.6 μg/ml NiCl2, CI14=3.3 μg/ml NiCl2, CI15=1.6 μg/ml SrCl2, CI16=3.3 μg/ml SrCl2, CI17=10 μl/ml DMSO, CI18=30 μl/ml DMSO, CI19=50 μl/ml DMSO, CI20=0 μl/ml DMSO (control), STD1=5254 medium, STD2=5294 medium, STD3=5567 medium, STD4=5429 medium.

FIG. 5 is a bar chart showing percentage of different culture conditions of extracts that showed >50% activity against the assay strain Pseudomonas aeruginosa ATCC 27853. MI=Actinobacteria strains that were cultivated in cultivation media where different microbial inducers were added, CI=Actinobacteria strains that were cultivated in cultivation media where different chemical inducers were added, STD=Actinobacteria strains that were cultivated in cultivation media.

FIG. 6 is a bar chart showing percentage of different microbial/chemical inducers and cultivation media of the extracts that showed >50% activity against the assay strain Pseudomonas aeruginosa ATCC 27853. MI1=supernatant of Staphylococcus aureus ATCC 33592 cell culture, MI2=supernatant of Escherichia coli ATCC 35218 cell culture, MI3=supernatant of Pseudomonas aeruginosa ATCC 27853 cell culture, MI4=supernatant of Candida albicans ATCC 753 cell culture, MI5=cells of Staphylococcus aureus ATCC 33592, MI6=cells of Escherichia coli ATCC 35218, MI7=cells of Pseudomonas aeruginosa ATCC 27853, MI8=cells of Candida albicans ATCC 753, CI1=1.6 μg/ml AsI3, CI2=3.3 μg/ml AsI3, CI3=1.6 μg/ml Pb(NO3)2, CI4=3.3 μg/ml Pb(NO3)2, CI5=1.6 μg/ml CdCl2, CI6=3.3 μg/ml CdCl2, CI7=1.6 μg/ml CoCl2, CI8=3.3 μg/ml CoCl2, CI9=1.6 μg/ml NaN3, CI10=3.3 μg/ml NaN3, CI11=1.6 μg/ml NaHSeO3, CI12=3.3 μg/ml NaHSeO3, CI13=1.6 μg/ml NiCl2, CI14=3.3 μg/ml NiCl2, CI15=1.6 μg/ml SrCl2, CI16=3.3 μg/ml SrCl2, CI17=10 μl/ml DMSO, CI18=30 μl/ml DMSO, CI19=50 μl/ml DMSO, CI20=0 μl/ml DMSO (control), STD1=5254 medium, STD2=5294 medium, STD3=5567 medium, STD4=5429 medium.

FIG. 7 is a bar chart showing percentage of different culture conditions of extracts that showed >50% activity against the assay strain Staphylococcus aureus ATCC 33592. MI=Actinobacteria strains that were cultivated in cultivation media where different microbial inducers were added, CI=Actinobacteria strains that were cultivated in cultivation media where different chemical inducers were added, STD=Actinobacteria strains that were cultivated in cultivation media.

FIG. 8 is a bar chart showing percentage of different microbial/chemical inducers and cultivation media of the extracts that showed >50% activity against the assay strain Staphylococcus aureus ATCC 33592. MI1=supernatant of Staphylococcus aureus ATCC 33592 cell culture, MI2=supernatant of Escherichia coli ATCC 35218 cell culture, MI3=supernatant of Pseudomonas aeruginosa ATCC 27853 cell culture, MI4=supernatant of Candida albicans ATCC 753 cell culture, MI5=cells of Staphylococcus aureus ATCC 33592, MI6=cells of Escherichia coli ATCC 35218, MI7=cells of Pseudomonas aeruginosa ATCC 27853, MI8=cells of Candida albicans ATCC 753, CI1=1.6 μg/ml AsI3, CI2=3.3 μg/ml AsI3, CI3=1.6 μg/ml Pb(NO3)2, CI4=3.3 μg/ml Pb(NO3)2, CI5=1.6 μg/ml CdCl2, CI6=3.3 μg/ml CdCl2, CI7=1.6 μg/ml CoCl2, CI8=3.3 μg/ml CoCl2, CI9=1.6 μg/ml NaN3, CI10=3.3 μg/ml NaN3, CI11=1.6 μg/ml NaHSeO3, CI12=3.3 μg/ml NaHSeO3, CI13=1.6 μg/ml NiCl2, CI14=3.3 μg/ml NiCl2, CI15=1.6 μg/ml SrCl2, CI16=3.3 μg/ml SrCl2, CI17=10 μl/ml DMSO, CI18=30 μl/ml DMSO, CI19=50 μl/ml DMSO, CI20=0 μl/ml DMSO (control), STD1=5254 medium, STD2=5294 medium, STD3=5567 medium, STD4=5429 medium.

FIG. 9 is a bar chart showing percentage of different culture conditions of extracts that showed >50% activity against the assay strain Candida albicans ATCC 753. MI=Actinobacteria strains that were cultivated in cultivation media where different microbial inducers were added, CI=Actinobacteria strains that were cultivated in cultivation media where different chemical inducers were added, STD=Actinobacteria strains that were cultivated in cultivation media.

FIG. 10 is a bar chart showing percentage of different microbial/chemical inducers and cultivation media of the extracts that showed >50% activity against the assay strain Candida albicans ATCC 753. MI1=supernatant of Staphylococcus aureus ATCC 33592 cell culture, MI2=supernatant of Escherichia coli ATCC 35218 cell culture, MI3=supernatant of Pseudomonas aeruginosa ATCC 27853 cell culture, MI4=supernatant of Candida albicans ATCC 753 cell culture, MI5=cells of Staphylococcus aureus ATCC 33592, MI6=cells of Escherichia coli ATCC 35218, MI7=cells of Pseudomonas aeruginosa ATCC 27853, MI8=cells of Candida albicans ATCC 753, CI1=1.6 μg/ml AsI3, CI2=3.3 μg/ml AsI3, CI3=1.6 μg/ml Pb(NO3)2, CI4=3.3 μg/ml Pb(NO3)2, CI5=1.6 μg/ml CdCl2, CI6=3.3 μg/ml CdCl2, CI7=1.6 μg/ml CoCl2, CI8=3.3 μg/ml CoCl2, CI9=1.6 μg/ml NaN3, CI10=3.3 μg/ml NaN3, CI11=1.6 μg/ml NaHSeO3, CI12=3.3 μg/ml NaHSeO3, CI13=1.6 μg/ml NiCl2, CI14=3.3 μg/ml NiCl2, CI15=1.6 μg/ml SrCl2, CI16=3.3 μg/ml SrCl2, CI17=10 μl/ml DMSO, CI18=30 μl/ml DMSO, CI19=50 μl/ml DMSO, CI20=0 μl/ml DMSO (control), STD1=5254 medium, STD2=5294 medium, STD3=5567 medium, STD4=5429 medium.

FIG. 11 shows a plot of the inhibition against the assay strain Candida albicans ATCC 753 of 79 fractions obtained after co-incubation of the recipient strain HAG012128 with the inducer strain Pseudomonas aeruginosa ATCC 27853, preparation of an extract, HPLC-separation of the extract into 79 fractions, re-collection and re-testing.

FIG. 12 shows a plot of TIC of positive MS-trace obtained by HPLC-MS showing the induced products dinactin (at 13.5 min) and trinactin (at 16.5 min) produced by strain HAG012128. FIG. 12A shows the chromatogram of the control reaction (standard medium 5294) and FIG. 12B shows the chromatogram of the co-incubation experiment, wherein strain HAG012128 is incubated with the inducer strain Pseudomonas aeruginosa ATCC 27853. For establishing the chromatogram, the whole extract of the co-incubation experiment was used.

### DETAILED DESCRIPTION

Silent genes are needed in most cases when microorganisms interact with other microorganisms. Based upon this, the present inventors have developed an approach in which such interactions are imitated in vitro. For this, recipient microorganisms are cultivated in the presence of killed microorganisms or inactivated culture supernatants of microorganisms with which the recipient microorganisms may be in contact in nature. By cultivating recipient microorganisms in the presence of killed microorganisms or inactivated culture supernatants, activation of silent genes is mediated by virtue of direct contact between the cells and/or by the action of messenger compounds.

In a first aspect, the present invention relates to a method for activation of silent genes comprising co-cultivation of a recipient microorganism and an inducer that activates silent genes in the recipient microorganism, wherein the inducer is selected from a chemical inducer and/or a microorganism inducer that is selected from a killed microorganism cell and/or inactivated culture medium in which the microorganism cell had been cultured.

In a second aspect, the present invention relates to a method for screening for an inducer that activates silent genes in a recipient microorganism, the method comprising the steps of:

(a) cultivating a recipient microorganism in the presence of a candidate inducer, and

(b) determining the candidate inducer as being an inducer, if silent genes are activated in the recipient microorganism,

wherein the candidate inducer is selected from a candidate chemical inducer and/or a candidate microorganism inducer that is selected from a killed microorganism cell and/or inactivated culture medium in which the microorganism cell had been cultured.

In a third aspect, the present invention relates to a method for screening for a recipient microorganism comprising the steps of:

(a) cultivating a candidate recipient microorganism in the presence of an inducer that activates silent genes in the recipient microorganism, and

(b) determining the candidate recipient microorganism as being a recipient microorganism if silent genes are activated in the candidate recipient microorganism,

wherein the inducer is selected from a chemical inducer and/or a microorganism inducer that is selected from a killed microorganism cell and/or inactivated culture medium in which the microorganism cell had been cultured.

The second and third aspects of the present invention may be regarded as being specific embodiments of the first aspect of the present invention.

The term “recipient microorganism” is meant in the present invention to include any microorganism. A microorganism is a microscopic organism that comprises either a single cell (unicellular) or cell clusters. Microorganisms are very diverse. They include bacteria, fungi, archaea, and protists; microscopic plants (green algae); and animals such as plankton and the planarian. The microorganism is capable of reacting to the presence of an “inducer” by the activation of silent genes. In a preferred embodiment, the term “recipient microorganism” refers to bacteria and fungi that are capable of reacting to the presence of an “inducer” by the activation of silent genes. A “candidate recipient microorganism” is a potential recipient microorganism because it is not known whether there is a silent gene therein that can be activated by an inducer, but which is tested therefor. The present invention provides a method for identifying a candidate recipient microorganism as a recipient microorganism.

The selection of suitable or candidate recipient microorganisms may be performed depending on various characteristics of a microorganism, which may be morphology or chemotaxonomy, which is the attempt to classify and identify organisms according to demonstrable differences and similarities in their biochemical compositions, genome information or MALDI-TOF analysis of protein patterns. The selection may also be performed by co-incubation of a microorganism that is tested for its capability as a recipient microorganism with another microorganism, such as a pathogenic microorganism or soil microorganism, especially a pathogenic or soil bacterium of the genus Acetobacter, Actinobacillus, Actinomadura, Actinomyces, Actinoplanes, Aeromonas, Alcaligenes, Alteromonas, Amycolatopsis, Arthrobacter, Aureobacterium, Bacillus, Bacteroides, Bifidobacterium, Borella, Brevibacterium, Burkholderia, Campylobacter, Cellulomonas, Clavibacter, Clostridium, Corynebacterium, Enterobacter, Enterococcus, Escherichia, Eubacterium, Flavobacterium, Fusobacterium, Haemophilus, Helicobacter, Klebsiella, Lactobacillus, Legionella, Microbacterium, Micrococcus, Micromonospora, Moraxella, Mycobacterium, Mycoplasma, Myxococcus, Neisseria, Nocardia, Pasteurella, Photorhabdus, Polyangium, Propionibacterium, Preoteus, Pseudomonas, Rhodococcus, Salmonella, Selenomonas, Serratia, Shigella, Sphingomonas, Staphylococcus, Streptococcus, Streptomyces, Thermoactinomyces, Treponema, Tsukamurella, Vibrio, Xanthomonas, Xenorhabdus or Yersinia or a fungus of the Ascomycota, Basidiomycota, Oomycota, Zygomycota, or yeasts in a medium, and by investigating whether the microorganism tested shows changes in phenotype versus a control using the same medium, however, without the other microorganism.

Matrix-assisted laser desorption/ionization (MALDI) is a soft ionization technique used in mass spectrometry, allowing the analysis of biomolecules (biopolymers such as DNA, proteins, peptides and sugars) and large organic molecules (such as polymers, dendrimers and other macromolecules) that tend to be fragile and fragment when ionized by more conventional ionization methods. MALDI is a two step process. First, desorption is triggered by a UV laser beam. Matrix material heavily absorbs UV laser light, leading to the ablation of the upper layer (˜micron) of the matrix material. A hot plume produced during the ablation contains many species: neutral and ionized matrix molecules, protonated and deprotonated matrix molecules, matrix clusters and nanodroplets. The second step is ionization (more accurately protonation or deprotonation). Protonation (deprotonation) of analyte molecules takes place in the hot plume. Some of the ablated species participate in protonation (deprotonation) of analyte molecules. The type of a mass spectrometer most widely used with MALDI is the TOF (time-of-flight mass spectrometer), mainly due to its large mass range. The TOF measurement procedure is also ideally suited to the MALDI ionization process since the pulsed laser takes individual ‘shots’ rather than working in continuous operation. The MALDI-TOF instrument is equipped with an ion mirror that reflects ions using an electric field, thereby doubling the ion flight path and increasing the resolution.

An “inducer”, as defined herein, is capable of initiating an event that results in the activation of a silent gene, which is then transcribed and translated into a protein. In a preferred embodiment, activation of a silent gene leads to a visible modulation of the phenotype of the recipient microorganism. The inducer may directly activate a silent gene, e.g. by directly activating the promoter, or may indirectly activate a silent gene, e.g., by activating other factors that act on the promoter. A “candidate inducer” is a potential inducer because it is not known whether it is capable of activating a silent gene in a recipient microorganism, but which is tested for that function. The present invention provides a method for identifying a candidate inducer as an inducer.

The term “silent gene” is meant to include genes that are in a non-coding state and do not encode a polypeptide. They are phenotypically silent DNA sequences not normally expressed during the life cycle of an individual, but are capable of activation. In a preferred embodiment of the present invention, a silent gene remains silent and is not activated if the microorganism is cultivated in standard media, which are used for the production of secondary metabolites containing complex C and N sources like soymeal, oatmeal, starch and peptone. A silent gene can be activated by adding into a standard medium one or several inducers in particular concentrations, mixtures or formulations. Such a silent gene inducer can be, for example, a small organic compound, a biomolecule (nucleotide or derivative, nucleic acid or derivative, protein, carbohydrate or derivate, polysaccharide, pharmaceutical compound, lysate of another microorganism or other biological material including tissues or organs, microbial organisms whether inactivated or alive). The term “silent gene” is meant to include genes that are in a non-coding state and do not encode a polypeptide. They are phenotypically silent DNA sequences not normally expressed during the life cycle of an individual, but capable of activation. Silent genes may be activated by any kind of physical stress, such as temperature or pressure different from standard conditions or any kind of chemical stress that is induced by life threatening compounds, such as toxins or heavy metals.

The term “activation of (activating) a silent gene” is meant to include a process of waking up a silent gene and transcribing its DNA. Such process usually requires many coordinated processes. Thus, the gene must be exposed to transcription factors, which must then pile onto specialized sequences adjacent to the gene that are called enhancer and promoter regions, which then attract RNA polymerase (the enzyme that catalyzes the synthesis of messenger RNA), which can then attach and prepare to read the gene's sequence.

In a fourth aspect of the present invention, the activation of silent genes results in a change of a phenotype of the recipient microorganism, such as change of production of metabolites, change of growth, change of morphology, and/or change of behaviour.

The term “phenotype” denotes characteristics or traits of the recipient microorganism, such as its morphology, development, biochemical or physiological properties or behaviour, which can be made visible by technical procedures. The term phenotype does not only include characteristics or traits that are visible in the appearance of the microorganism, such as growth, morphology or behaviour, but includes hidden characteristics or traits that are not visible if looking at the microorganism, but which can be made visible. Such characteristics or traits include the presence or absence or changed amounts of chemical substances, such as metabolites. A change of the phenotype includes the visible change of the appearance of a microorganism, such as the increase or decrease of growth of the microorganism or the change of the morphology or of the behavior, such as motility. Thus, in one embodiment of the present invention, a phenotype as comprised by the present invention that indicates an interaction of a recipient microorganism and an inducer and thus the activation of silent genes refers to the amount of a metabolite, whereby the amount of the metabolite may be increased or decreased. One such metabolite may be ATP (adenosine triphosphate). Preferably, the amount of a metabolite, e.g., one that participates in constitutive pathways of a cell such as ATP produced by the recipient microorganism, is diminished by the activity of an inducer. If the metabolite is a secondary metabolite, such metabolites are usually not produced by the recipient strain, but are produced upon contact of the recipient microorganism with an inducer. In this case the change of a phenotype results in the increase of the amount of the metabolite. Other phenotypes as comprised by the present invention refer to growth, whereby the growth of the recipient microorganisms may be enhanced or inhibited, morphology, or behaviour. It is understood by the person skilled in the art that, e.g., reduction of the amount of a metabolite, such as ATP, may be accompanied by growth inhibition.

Consequently, in the context of the present invention, an inducer effects a change of a phenotype of the recipient microorganism. An inducer may result in the change of the amount of a metabolite. The inducer may result in the decrease of the amount of a metabolite, e.g., of a metabolite that participates in constitutive pathways of a cell, such as ATP. The inducer may result in the increase of the amount of a secondary metabolite, e.g., metabolites that are usually not produced by the recipient strain, but are produced upon contact of the recipient microorganism with an inducer. Inducers in the context of the present invention are also inducers that change, preferably inhibit, the growth of a recipient microorganism, change the morphology or behavior of the recipient microorganism.

The effect of an inducer on a recipient microorganism can be directly determined. Thereby, the change of the phenotype is directly determined with the recipient microorganism, e.g., by directly determining the amount of a metabolite, the growth, morphology or behavior of the recipient microorganism. The effect of an inducer on a recipient microorganism can also be determined indirectly, for example, by determining the effects of an induced recipient microorganism on an assay strain. An assay strain, assay microorganism or assay cell, as used mutually herein, is a strain used for detecting whether a silent gene in a recipient microorganism has been induced by an inducer. Thereby, the cells of the induced recipient microorganism, the supernatant of the co-cultivation medium of the recipient microorganism and inducer or an extract of the supernatant are cultivated with the assay strain and the effects thereof on the assay strain are investigated. Cells and supernatant are prepared by methods known in the art, such as centrifugation, filtration, flocculation and/or precipitation. The extracts are either derived from the supernatant or may be the supernatant (polar extracts) or are prepared by concentration of the supernatant by any method known in the art including evaporation, vacuum concentration, lyophilization, reverse extraction, solute precipitation and dialysis, preferably lyophilization (non-polar extracts). The extracts can be resolved in an aqueous or organic solution. Possibly, the resolved concentrate may be adsorbed to a resin, preferably a ion exchange resin, such as HP 20, and eluted. Purification is achieved by state of the art chromatography systems, e.g., HPLC (as disclosed e.g. in: HPLC richtig optimiert; ed: Stavros Kromidas; Wiley 2006). Preferably, extracts are used. More preferably, non-polar extracts are used. If the inducer results in the activation of a silent gene in a recipient microorganism, such effects can be determined by determination of the change of a phenotype in the assay strain. The change of a phenotype of an assay strain may be due to the change of the production of one or more metabolites in the recipient strain, which one or more metabolites effect a change of a phenotype in the assay strain. The kinds of phenotype that are changed with the assay strain and that are investigated in the present invention are the same with respect to the recipient microorganism, namely the change of the amount of a metabolite, the change of growth, the change of morphology or the change of behavior. Thereby, the preferred phenotype is the change of a metabolite, more preferably the change of the amount of ATP, and still more preferably the decrease of the amount of ATP within the assay strain. The assay strain may be any microorganism that allows the detection of the effects of an induced recipient microorganism cell, supernatant or extract therefrom, preferably the assay strain is of the genus Escherichia, Staphylococcus, Pseudomonas or Candida, more preferably Escherichia coli, Staphylococcus aureus, Pseudomonas aeruginosa or Candida albicans, and most preferably Escherichia coli ATCC 35218, Staphylococcus aureus ATCC 33592, Pseudomonas aeruginosa ATCC 27853 or Candida albicans ATCC 753. These strains are publicly available from the American Type Culture Collection.

The inhibition of the assay strain with respect to productivity of a metabolite or growth is also referred to in the present invention as “inhibitory activity” of an inducer or inducer “activity against” the assay stain or cognate terms.

The terms “inhibition”, “inhibitory activity” and “activity against” or cognate terms are meant to inhibit the production of a metabolite, such as ATP, or growth in the assay strain by at least 5%, at least 10%, at least 20%, at least 30%, at least 40%, at least 50%, at least 60%, at least 80%, at least 90%, or 100% as compared to the production of the same metabolite or growth in the absence of an inducer. In a preferred embodiment, the production of the metabolite or growth of an assay strain is inhibited by at least 30%, more preferably by at least 40% and most preferably by at least 50%.

The term “selective inhibition”, “selective inhibitory activity” or “selective activity against” or cognate terms denote that an inducer inhibits the production of a metabolite or growth of a specific assay strain, whereas it does not have an inhibitory activity against another assay strain. In the context of the present invention, the term “selective inhibitory activity” or “selective activity against” means that an inducer inhibits the production of a metabolite or growth of one of Escherichia coli ATCC 35218, Staphylococcus aureus ATCC 33592, Pseudomonas aeruginosa ATCC 27853 or Candida albicans ATCC 753, whereas the growth of the other strains is not inhibited. In a preferred embodiment, the term “inhibition”, “inhibitory activity” or “activity against” “selective inhibition”, “selective inhibitory activity” or “selective activity against” or cognate terms refer to the inhibition of the production of ATP.

Metabolites are the intermediates or products of metabolism. A primary metabolite is directly involved in normal growth, development, and reproduction. Alcohol is an example of a primary metabolite. A secondary metabolite is not directly involved in those processes, but usually has an important ecological function. Unlike primary metabolites, absence of secondary metabolites does not result in immediate death, but rather in long-term impairment of the organism's survivability, fecundity, or aesthetics, or perhaps in no significant change at all. Secondary metabolites often play an important role in plant defense against herbivory and other interspecies defenses. Humans use secondary metabolites as medicines, flavorings, and recreational drugs. Secondary metabolites may be classified based on their biosynthetic origin. Such classes include alkaloids, terpenoids, steroids, glycosides, glucosinolates, phenazines, polyketides, fatty acid synthase products, nonribosomal peptides and ribosomal peptides. Metabolites whose change in amount indicates the activation of a silent gene and whose detection is therefore useful in the methods of the present invention are characterized by, e.g., additional output of biochemical assays (e.g., additional peaks in spectrograms) or additional activity in biological test systems (e.g., growth inhibition of bacteria, fungi or tumor tissue).

A variety of assays are known in the art to detect metabolites that are produced in a recipient cell or assay cell in reaction to an inducer. In a preferred embodiment, the activity of an inducer is detected by measuring the amount of ATP produced by an assay strain. This may be done by any method known in the art for measuring ATP. One such method is the use of the BacTiter-Glo™ assay. The BacTiter-Glo™ microbial cell viability assay is a homogenous method for determining the number of viable bacterial cells in culture based on quantitation of the ATP present. ATP is an indicator of metabolically active cells. The BacTiter-Glo™ assay is designed to be used in a multiwell-plate format. The homogenous assay procedure involves adding a single reagent (BacTiter-Glo™ reagent) directly to bacterial cells in medium measuring luminescence (DeLuca and McElroy W. D. (1978), McElroy and DeLuca (1983).

Another method for detection of metabolites is mass spectrometry after separation by GC (gas chromatography), HPLC (high-performance liquid chromatography) (LC-MS), or CE (capillary electrophoresis). The term “mass spectrometry” refers to the use of an ionization source to generate gas phase ions from a sample on a surface and detecting the gas phase ions with a mass spectrometer. In mass spectrometry the “apparent molecular mass” refers to the molecular mass (in Daltons)-to-charge value, m/z, of the detected ions.

Traditionally, detection of common or expected metabolites has been conducted on LC/MS data by generating extracted or reconstructed ion chromatograms corresponding to the expected protonated molecules of drug metabolites. In liquid chromatography-mass spectrometry (LC-MS, or alternatively HPLC-MS), the physical separation capabilities of liquid chromatography (or HPLC) is combined with the mass analysis capabilities of mass spectrometry. LC-MS is a powerful technique used for many applications, which has very high sensitivity and selectivity. Generally its application is oriented towards the general detection and potential identification of chemicals in the presence of other chemicals (in a complex mixture). Many different mass analyzers can be used in LC/MS, such as Single Quadrupole, Triple Quadrupole, Ion Trap, TOF (time of Flight), and Quadrupole-time of flight (Q-TOF). Over the last decade, product ion scanning techniques that use rule-based algorithms to generate a list of potential metabolite masses have been developed and continuously improved for rapid screening for common metabolites. The technique employs a survey mode to search for the metabolites that are listed in the acquisition method. Both the detection of expected metabolites and the acquisition of their product ion spectra can be accomplished in a single LC/MS analysis. With the availability of comprehensive metabolite databases developed from knowledge of biotransformations, the list-dependent product ion scan has been very successful in screening for predicted metabolites, especially in vitro metabolites.

Detection of uncommon metabolites in complex biological matrices is more challenging, and is often carried out using precursor ion (PI) or neutral loss (NL) scanning techniques on a triple quadrupole mass spectrometer. The detection of conjugates (e.g., glucuronide and sulfate) can usually be accomplished with an NL analysis because these conjugates often undergo common cleavages to generate specific neutral fragments under collision-induced dissociation conditions. PI scanning can also be used to search for metabolites with common product ions that can be predicted from the patterns of the parent drug product ions. For a PI or NL analysis, however, one or a few expected neutral or charged fragments must be defined in a LC/MS/MS acquisition method. Metabolites that do not generate the expected fragments will not be detected.

The task of metabolite identification has been greatly facilitated by recent developments in high resolution LC/MS technology (e.g., time-of-flight (ToF) and Fourier transform (FT) mass spectrometers), which allow for the determination of molecular formulae and product ion formulae with minimal uncertainty. In addition, the specificity of list-dependent acquisition of MS/MS data for expected metabolites is improved. Similarly, triple quadrupole mass spectrometry with improved mass resolution has provided improved selectivity in NL and PI analyses. A combination of high resolution mass spectrometry and other types of LC/MS instruments has been recommended for metabolite identification, given the complementary capabilities of triple quadrupole, ion trap, and high resolution mass spectrometers.

High-pressure liquid chromatography-atmospheric pressure ionization mass spectrometry (LC-API-MS) is a powerful means for separation, detection, and identification of products from xenobiotic metabolism. With the commercial introduction of new ionization methods, such as those based on atmospheric pressure ionization (API) techniques and the combination of liquid chromatography-mass spectrometry (LC-MS), it has now become a truly indispensable technique in pharmaceutical research. Triple stage quadrupole and ion trap mass spectrometers are presently used for this purpose, because of their sensitivity and selectivity. API-TOF mass spectrometry has also been very attractive due to its enhanced full-scan sensitivity, scan speed, improved resolution and ability to measure the accurate masses for protonated molecules and fragment ions.

In addition, mass spectral fingerprint libraries exist or can be developed that allow identification of a metabolite according to its fragmentation pattern.

Surface-based mass analysis has seen a resurgence in the past decade, with new MS technologies focused on increasing sensitivity, minimizing background, and reducing sample preparation. The ability to analyze metabolites directly from biofluids and tissues continues to challenge current MS technology, largely because of the limits imposed by the complexity of these samples, which contain thousands to tens of thousands of metabolites. Among the technologies being developed to address this challenge is Nanostructure-Initiator MS (NIMS), a desorption/ionization approach that does not require the application of matrix and thereby facilitates small-molecule (i.e., metabolite) identification. MALDI is also used, however, the application of a MALDI matrix can add significant background at <1000 Da that complicates analysis of the low-mass range (i.e., metabolites). In addition, the size of the resulting matrix crystals limits the spatial resolution that can be achieved in tissue imaging. Because of these limitations, several other matrix-free desorption/ionization approaches have been applied to the analysis of biofluids and tissues. Secondary ion mass spectrometry (SIMS) was one of the first matrix-free desorption/ionization approaches used to analyze metabolites from biological samples. SIMS uses a high-energy primary ion beam to desorb and generate secondary ions from a surface. The primary advantage of SIMS is its high spatial resolution (as small as 50 nm), a powerful characteristic for tissue imaging with MS. However, SIMS has yet to be readily applied to the analysis of biofluids and tissues because of its limited sensitivity at >500 Da and analyte fragmentation generated by the high-energy primary ion beam. Desorption electrospray ionization (DESI) is a matrix-free technique for analyzing biological samples that uses a charged solvent spray to desorb ions from a surface. Advantages of DESI are that no special surface is required and the analysis is performed at ambient pressure with full access to the sample during acquisition. A limitation of DESI is spatial resolution because “focusing” the charged solvent spray is difficult. However, a recent development termed laser ablation ESI (LAESI) is a promising approach to circumvent this limitation.

Another widely used method for detecting metabolites is nuclear magnetic resonance (NMR) spectroscopy. NMR is the only detection technique that does not rely on separation of the analytes, and the sample can thus be recovered for further analyses. All kinds of small molecule metabolites can be measured simultaneously. Thus, NMR is close to being a universal detector. The main advantages of NMR are high analytical reproducibility and simplicity of sample preparation. NMR is a physical phenomenon in which magnetic nuclei in a magnetic field absorb and re-emit electromagnetic radiation. This energy is at a specific resonance frequency that depends on the strength of the magnetic field and the magnetic properties of the isotope of the atoms. NMR allows the observation of specific quantum mechanical magnetic properties of the atomic nucleus. Many scientific techniques exploit NMR phenomena to study molecular physics, crystals, and non-crystalline materials through NMR spectroscopy.

Although NMR and MS are the most widely used techniques, other methods of detection include ion-mobility spectrometry, electrochemical detection (coupled to HPLC) and radiolabel (when combined with thin-layer chromatography).

Other methods for detecting metabolites are immunobased methods, such as enzyme-linked immunosorbent assay (ELISA), or a relatively similar method, the enzyme immunoassay (EIA) to detect the presence of a substance in a liquid sample or wet sample. Performing an ELISA involves at least one antibody with specificity for a particular antigen. The sample with an unknown amount of antigen is immobilized on a solid support either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody. Between each step, the plate is typically washed with a mild detergent solution to remove any proteins or antibodies that are not specifically bound. After the final wash step, the plate is developed by adding an enzymatic substrate to produce a visible signal, which indicates the quantity of antigen in the sample. ELISA typically involves chromogenic reporters and substrates that produce some kind of observable color change to indicate the presence of antigen or analyte. ELISA-like techniques utilize fluorogenic, electrochemiluminescent, and real-time PCR reporters to create quantifiable signals. These new reporters can have various advantages including higher sensitivities and multiplexing.

In EIA, detection is not performed using a second labeled antibody, however, a labeled competitor antigen is used, resulting in competition of analyte and competitor for a binding site on the antibody.

The detection involves the use of specific antibodies. Such antibodies are either known in the art and available (for example commercially available), or can be raised using well established techniques for immunizing animals with prepared forms of the antigen. A variety of reagents is available to assist in antibody production and purification, and various companies specialize in antibody production services. Depending on the application to be performed, different levels of purity and types of specificity are needed in a supplied primary antibody. To name just a few parameters, antibodies may be monoclonal or polyclonal, supplied as antiserum or affinity-purified solution.

An antibody that recognizes the target metabolite is called the “primary antibody.” If this antibody is labeled with a tag, direct detection of the metabolite is possible. Usually, however, the primary antibody is not labeled for direct detection. Instead a “secondary antibody” that has been labeled with a detectable tag is applied in a second step to probe for the primary antibody, which is bound to the target antigen. Thus, the metabolite is detected indirectly. Another form of indirect detection involves using a primary or secondary antibody that is labeled with an affinity tag such as biotin. Then a secondary (or tertiary) probe, such as streptavidin that is labeled with the detectable enzyme or fluorophore tag, can be used to probe for the biotin tag to yield a detectable signal. Several variants of these probing and detection strategies exist. However, each one depends on a specific probe (e.g., a primary antibody) whose presence is linked directly or indirectly to some sort of measurable tag (e.g., an enzyme whose activity can produce a colored product upon reaction with its substrate).

Usually, a primary antibody without a detectable label and some sort of secondary (indirect) detection method is required in assay methods. Nevertheless, nearly any antibody can be labeled with biotin, HRP enzyme, or one of several fluorophores if needed. Most primary antibodies are produced in mouse, rabbit, or one of several other species. Nearly all of these are antibodies of the IgG class. Therefore, it is relatively easy and economical for manufacturers to produce and supply ready-to-use, labeled secondary antibodies for most applications and detection systems. Even so, several hundred options are available, differing in the level of purity, IgG- and species-specificity, and detection label. The choice of secondary antibody depends upon the species of animal in which the primary antibody was raised (the host species). For example, if the primary antibody is a mouse monoclonal antibody, then the secondary antibody must be an anti-mouse antibody obtained from a host other than the mouse.

The growth of a microorganism can be assayed by any method used in the art, whereby the kind of assessment depends on the selected recipient or assay microorganism. For microorganisms such as, e.g., bacteria, growth may be measured in terms of two different parameters: changes in cell mass and changes in cell numbers. Methods for measurement of the cell mass involve both direct and indirect techniques and include direct physical measurement of dry weight, wet weight, or volume of cells after centrifugation, direct chemical measurement of some chemical components of the cells, such as total N, total protein, or total DNA content, indirect measurement of chemical activity, such as rate of O2 production or consumption, CO2 production or consumption, ATP production, etc., and turbidity measurements, which employ a variety of instruments to determine the amount of light scattered by a suspension of cells. The turbidity or optical density of a suspension of cells is directly related to cell mass or cell number. Methods for measurement of cell numbers involve direct counts including viable cell counting, visually or instrumentally, and indirect viable cell counts. Direct microscopic counts are possible using special slides known as counting chambers. Dead cells cannot be distinguished from living ones. Only dense suspensions can be counted (>107 cells per ml). Electronic counting chambers count numbers and measure size distribution of cells. Indirect viable cell counts, also called plate counts, involve plating out (spreading) a sample of a culture on a nutrient agar surface. The sample or cell suspension can be diluted in a nontoxic diluent (e.g. water or saline) before plating. If plated on a suitable medium, each viable unit grows and forms a colony. Each colony that can be counted is called a colony forming unit (cfu) and the number of cfus is related to the viable number of bacteria in the sample. Determination of Candida albicans growth may be inter alia assessed by extracted mannan levels by an enzyme-linked immunosorbent assay, which method shows good correlation with fungal biomass (dry weight). Characterization of C. albicans growth may also be with respect to germ tube or chlamydospore production or sugar assimilation. A preferred method for determining the growth of a microorganism is by measuring the production of a metabolite such as ATP, which is an indirect measure for growth rate.

For detecting inhibition of growth of a recipient microorganism or an assay strain, the IC50 value is determined. The IC50 value is the concentration of a compound that is necessary to inhibit the growth of a test organism by 50%. The IC50 may be determined by any method known in the art. Thus, the IC50 value may be determined by measurement of the inhibition concentrations with the BacTiter-Glo™ Microbial Cell Viability Assay or by measurement of cell turbidity and the use of a program like XLfit and the corresponding formula.

The change of morphology is a directly visible trait and may inter alia be determined by viewing the recipient microorganism or assay strain.

The change of behavior may be a directly visible trait, such as a change of motility and may inter alia be determined by viewing the recipient microorganism or assay strain.

The activation of a silent gene in a recipient microorganism is determined in comparison to a reference or control. References or controls are a part of the test methods, since they can eliminate or minimize unintended influences (such as background signals). Controlled experiments are used to investigate the effect of a variable on a particular system. In a controlled experiment, one set of samples has been (or is believed to be) modified and the other set of samples is either expected to show no change (negative control) or expected to show a definite change (positive control). The control can be determined in one test run together with the test substance or under the test condition. It may be determined before or after determining the effect of the test compound or test condition or it may be a known value. A possible control experiment may be an experiment in which the same conditions are used as in the test experiment, however, the variant compound is used instead of the corresponding substance of the control assay. The reference or control medium may be a medium that is used to culture microorganisms and does not contain a candidate inducer or an inducer. Consequently, such medium does not activate silent genes in a recipient microorganism. In another embodiment, a control medium may comprise a component that is able to activate silent genes, which component is, however, not an inducer in the sense of the present invention, which is relevant for the present invention. For example, such component may be present in a medium without being known that such component activates silent genes. Or such component may be known to activate silent genes, however, it may be necessary for cultivating the recipient microorganism. Nevertheless, the activity of such component remains irrelevant for the purpose of the present invention, as, for determining the activity of an inducer, the control medium differs from the co-cultivation medium by the absence of the inducer, whereas the component is present in both the control medium and the co-cultivation medium. In the context of the first and second aspects of the present invention, a suitable control medium may be a medium that does not contain a component that activates a silent gene. In another embodiment, the control medium activates a silent gene due to the presence of a component that activates a silent gene. However, as the component is present in the control medium as well as in the test medium, any differences of activation of a silent gene can be traced back to the inducer that is additionally added to the control medium. The control medium is the same medium as used in the test method, however, does not comprise an inducer or candidate inducer. Activation of silent genes in the same medium comprising a candidate inducer, indicates that the candidate inducer is effective as an inducer. In the context of the third aspect of the present invention, the control medium is a medium in which the candidate recipient microorganism is able to grow. The test medium is the same medium as the control medium. To the test medium, an inducer is added so that the test medium additionally comprises the inducer, which is a known inducer or which is identified as an inducer e.g., according to the method provided herein, and screening is performed with various candidate recipient microorganisms. A candidate recipient microorganism in which silent genes are activated due to the presence of the added inducer is a recipient microorganism for said inducer. In one embodiment, the control medium itself does not activate silent genes. In another embodiment, the control medium activates a silent gene due to the presence of a component that activates a silent gene. However, as the component is present in the control medium as well as in the test medium, any differences of activation of a silent gene can be traced back to the inducer that is additionally added to the control medium. A control medium can be any standard medium as long as it does not contain the inducer or candidate inducer as, e.g., the Müller-Hinton medium (30% beef infusion; 1.75% casein hydrolysate; 0.15% starch; pH adjusted to neutral at 25° C.; percentage amounts as w/w), the Sabouraud medium for yeast growth (10 g/l polypeptone or neopeptone; 40 g/l dextrose; final pH about 5.8) or Nutrient broth for soil bacteria (0.5 peptone, 0.3% beef extract/yeast extract and 0.5% NaCl, final pH 6.8 at 25° C.). Other control media are medium 5254, medium 5294, medium 5567, or medium 5429. The composition of medium 5254 is as follows (amount in percent w/w): glucose 1.50, soybean meal 1.50, cornsteep 0.50, CaCO3 0.20, NaCl 0.50. The medium is sterilized for 20 minutes at 121° C. The pH value before sterilization is 7.00. The composition of medium 5294 is as follows (amount in percent w/w): soluble starch 1.00, glucose 1.00, 99% glycerin 1.00, cornsteep liquor 0.25, peptone 0.50, yeast extract 0.20, CaCO3 0.30, NaCl 0.10. The medium is sterilized for 20 minutes at 121° C. The pH value before sterilization is 7.20. The composition of medium 5567 is as follows (amount in percent w/w): oatmeal 2.00, Spur 5314 0.25, agar 1.80. The medium is sterilized for 30 minutes at 121° C. The pH value is 7.80 before sterilization and 7.20 after sterilization. The composition of medium 5429 is as follows (amount in percent w/w): glucose 0.40, yeast extract 0.40, malt extract 1.00, CaCO3 0.20. The medium is sterilized for 20 minutes at 121° C. The pH value is adjusted to 7.20 with KOH before sterilization. Further media of choice are the media as described in R.M. Atlas: Handbook of Microbiological Media; London: CRC Press 2004; ISBN 0849318181 and in Manual of Industrial Microbiology and Biotechnology By Arnold Demain and Julian Davies, American Society for Microbiology, 1999. A standard medium is any standard medium known in the art for cultivating bacteria and fungi, which comprises complex N- and/or C-sources such as soymeal, peptone, cornsteep etc. Medium 5254, medium 5294, medium 5567 and medium 5429 are standard media.

A growth medium or culture medium, as used herein, for growing or cultivating a recipient microorganism is a liquid or solid medium designed to support the growth of microorganisms. An important distinction between growth media types is that of defined (also synthetic) versus undefined (also basal or complex) media. A defined medium will have known quantities of all ingredients. For microorganisms, they consist of providing trace elements and vitamins required by the microbe and especially a defined carbon source and nitrogen source. Minimal media are those that contain the minimum nutrients possible for colony growth, generally without the presence of amino acids, and are often used by microbiologists and geneticists to grow “wild type” microorganisms. Selective media are used for the growth of only selected microorganisms. Differential media or indicator media distinguish one microorganism type from another growing on the same media. This type of media uses the biochemical characteristics of a microorganism growing in the presence of specific nutrients or indicators (such as neutral red, phenol red, eosin, or methylene blue) added to the medium to visibly indicate the defining characteristics of a microorganism. Enriched media contain the nutrients required to support the growth of a wide variety of organisms. All of these media are included within the present invention, as long as the selected microorganism can grow on it.

In principle, any medium that allows the growth of the recipient microorganisms used may be selected for the co-cultivation. Co-cultivation of the recipient microorganisms is carried out in a solid or liquid medium. In a preferred embodiment, the co-cultivation is carried out in an aqueous solution, wherein the medium comprises components necessary to allow growth of the recipient microorganism. The skilled person thereby knows or is capable of identifying those components that are necessary for the growth of the microorganism. The co-cultivation is carried out at a pH of 2 to 10 depending on the recipient microorganism, preferably at a pH of 4 to 8, and more preferably at a pH of 6 to 8. Optionally, the samples may be incubated at a temperature suitable for the growth of the recipient microorganism, preferably at temperatures between 0 and 50° C., more preferably at temperatures between 10 and 40° C., even more preferably between temperatures between 20 and 40° C., and most preferably at 30° C. Typically, the reaction duration is between 10 and 250 hours, preferably 30 to 200 hours, and more preferably 45 to 170 hours. The reaction time depends on the microorganism used. Advantageous and optimal reaction times can be easily determined by those skilled in the art.

The medium should comprise any nutrients that are necessary for the growth of the microorganism. Essential nutrients comprise assimilable carbon sources, assimilable nitrogen sources, and minerals, and, if necessary, growth factors.

As assimilable carbon sources, a series of carbohydrates may be used, as long as they can be used by the microorganism. Useable carbon sources are glucose, sucrose, lactose, dextrins, starch, molasses, or sugar alcohols, such as glycerol, mannitol, or sorbitol. A preferred carbohydrate source is sucrose. The carbohydrates are present altogether, preferably in an amount of 5 to 30 g/l, more preferably in an amount of 10 to 25 g/l, most preferably in an amount of 12 to 20 g/l.

As assimilable nitrogen sources, substances such as nitrate, anorganic or organic ammonium salts, urea and amino acids, or more complex substances, such as proteins, such as casein, lactalbumin, gluten or the hydrolysates thereof or soybean flour, fish meal, meat extract, yeast extract, distillers' soluble, corn steep liquor or corn steep solid may be used. The nitrogen sources are present altogether preferably in an amount of 5 to 30 g/l, more preferably in an amount of 10 to 25 g/l, and most preferably in an amount of 12 to 20 g/l.

As minerals, alkali or earth alkali salts, such as alkali or earth alkali chloride, carbonate, phosphate or sulfate are usable. Examples of alkali or earth alkali metals are sodium, calcium, zinc, cobalt, iron, copper and manganese salts. The salts are preferably present altogether in an amount of 5 to 25 g/l.

If necessary for the growth of a microorganism, other factors may be included. The skilled person knows or will be able to elucidate which factors are to be used to cultivate a selected microorganism.

Typical media include Mueller Hinton broth for pathogenic bacteria, Sabouraud for yeasts and fungi and Nutrient broth for soil bacteria, medium 5254, medium 5294, medium 5567 or medium 5429 or media as described in R.M. Atlas: Handbook of Microbiological Media; London: CRC Press 2004; ISBN 0849318181 and in Manual of Industrial Microbiology and Biotechnology By Arnold Demain and Julian Davies, American Society for Microbiology, 1999.

The disclosure with respect to growth or culture medium and cultivating conditions of the recipient microorganisms also apply to the assay strain.

The growth or culture medium referred to above is a medium useful for cultivating a recipient microorganism under non-induction conditions. In the methods comprised by the present invention, an inducer or candidate inducer is added to such medium to result in the activation of a silent gene of the recipient microorganism. The amount of inducer is dependent on the inducer and the recipient microorganism. The skilled person will be able to determine the amount of inducer that results in activation of a silent gene. If the inducer is an inactivated culture medium in which the inducer microorganism had been cultured, then cultivation of the recipient microorganism takes place in the inactivated culture medium. Cultivation of a recipient microorganism in the presence of an inducer is referred to herein as co-cultivation.

The co-cultivation can be carried out in a microscale, e.g. in microtiter plates with a scale of 10 μl to 1200 μl, or in the scale of shake flask cultivation with a scale of 5 ml to 500 ml or in the scale of bioreactors with a scale of at least 501. The scale is therefore in the range of some microliters to thousands of liters such as 10 μl to 1,000,000 liter.

In an embodiment of the invention, the inducer is a chemical inducer. The term “chemical inducer” relates to any chemical that is suitable to activate silent genes. A chemical inducer may be selected from the group consisting of a nucleic acid, a peptide or protein, an amino acid, an organic or anorganic salt, a metabolite, or a low molecular weight compound (LMW). LMWs are molecules that are, by definition, not a polymer and are not proteins, peptide antibodies, polysaccharides or nucleic acids. Very small oligomers are usually considered small molecules, such as dinucleotides, peptides, and disaccharides. LMWs comprise drugs, primary and secondary metabolites, such as alkaloids, glycosides, lipids, flavonoids, nonribosomal peptides, phenazines, phenols, polyketides, terpenes, or tetrapyrroles. They exhibit a molecular weight of less then 2000 Da and more preferably less than 800 Da. Such LMWs may be identified in high-through-put procedures starting from libraries. Libraries or collections are commercially available. Chemical inducers are in a preferred embodiment CoCl2, SrCl2, NaHSeO3 CdCl2, AsI3, NiCl2, Pb(NO3)2, NaN3.

A chemical inducer may be used alone for activating silent genes. Alternatively, a combination of one, two, three, or more other chemical inducers may be used to activate silent genes. The chemical inducer is present in the co-cultivating medium in a concentration suitable to activate silent genes in the recipient microorganism, as, e.g., expressed by the inhibition of the production of a metabolite, such as ATP, in the recipient or preferably assay strain. Suitable concentrations depend on the chemical inducer and the recipient microorganism. The skilled person will be capable of determining the concentration at which a chemical inducer is capable of activating a silent gene. The extent of inhibition is as indicated above. The chemical inducers are preferably used in the range of 0.001 to 1 mg/l, more preferably in the range of 0.001 to 0.1 mg/l, and still more preferably in the range of 0.001 to 0.05 mg/l medium. If the inducer is per se liquid, such as DMSO, the concentration is preferably in the range of 0.1 μl/ml to 1000 μl/ml, more preferably 1 μl/ml to 100 μl/ml, and still more preferably 10 μl/ml to 50 μl/ml medium.

In another embodiment, the inducer is a microorganism inducer that is selected from a killed microorganism and/or inactivated culture medium, in which the microorganism had been cultured. A microorganism inducer is derived from any microorganism as long as the inducer is able to activate silent genes in a recipient microorganism. The microorganisms may be bacteria or fungi. The bacteria or fungi may be selected from the genus Acetobacter, Actinobacillus, Actinomadura, Actinomyces, Actinoplanes, Aeromonas, Alcaligenes, Alteromonas, Amycolatopsis, Arthrobacter, Aureobacterium, Bacillus, Bacteroides, Bifidobacterium, Borella, Brevibacterium, Burkholderia, Campylobacter, Cellulomonas, Clavibacter, Clostridium, Corynebacterium, Enterobacter, Enterococcus, Escherichia, Eubacterium, Flavobacterium, Fusobacterium, Haemophilus, Helicobacter, Klebsiella, Lactobacillus, Legionella, Microbacterium, Micrococcus, Micromonospora, Moraxella, Mycobacterium, Mycoplasma, Myxococcus, Neisseria, Nocardia, Pasteurella, Photorhabdus, Polyangium, Propionibacterium, Preoteus, Pseudomonas, Rhodococcus, Salmonella, Selenomonas, Serratia, Shigella, Sphingomonas, Staphylococcus, Streptococcus, Streptomyces, Thermoactinomyces, Treponema, Tsukamurella, Vibrio, Xanthomonas, Xenorhabdus or Yersinia or the Ascomycota, Basidiomycota, Oomycota, Zygomycota or yeasts. Of these microorganisms genus Escherichia, Staphylococcus, Pseudomonas, or Candida are preferred. For obtaining an inducer selected from a killed microorganism or an inactivated culture medium, the microorganism is cultivated. Cultivation depends on the type of microorganism. The conditions for culturing a specific microorganism are known to those skilled in the art. In principle, the conditions are those as referred to above with respect to the co-cultivation conditions. The media may be those as referred to above with respect to the recipient microorganism cultured under non-induction conditions. In a preferred embodiment, the medium for culturing the microorganisms is Müller-Hinton medium, Sabouraud medium or Nutrient broth. Müller-Hinton medium is especially preferred for growing inducer strains of the genus Escherichia, such as Escherichia coli such as Escherichia coli ATCC 35218, strains of the genus Staphylococcus, such as Staphylococcus aureus such as Staphylococcus aureus ATCC 33592, strains of the genus Pseudomonas, such as Pseudomonas aeruginosa such as Pseudomonas aeruginosa ATCC 27853 or strains of the genus Candida, such as Candida albicans such as Candida albicans ATCC 753. Other examples for a typical medium for growing E. coli is a medium known in the art as LB Medium or L-Broth, which typically contains 10 g of tryptone and 5 g of yeast extract per liter, and can vary in salt concentration from 0.5 g to 10 g per liter. A typical medium for growing S. aureus is nutrient broth or nutrient agar. P. aeruginosa has very simple nutritional requirements. It is often observed growing in distilled water, which is evidence of its minimal nutritional needs. In the laboratory, a typical medium for growth of P. aeruginosa consists of acetate as a source of carbon and ammonium sulfate as a source of nitrogen. Organic growth factors are not required, and it can use more than 75 organic compounds for growth. Exemplary media for growing C. albicans are PDA (potato dextrose agar) or FSA (fungal selection agar).

After cultivation, the microorganism cells are separated from the medium by any methods known in the art, to result in the microorganism cells and the culture medium. These may be centrifugation, filtration, flocculation and/or precipitation.

The cultured cells of the microorganism may be killed by physical and/or chemical means. Physical means is by heat such as dry heat, wet heat (autoclaves), tyndallisation or pasteurization or by irradiation. The kind, temperature and length of heat application depends on the microorganism used as inducer. In general, dry heat is less effective than moist heat. For example, spores of Clostridium botulinum are killed in saturated steam in five minutes at 120° C., while it takes two hours at 160° C. in a dry air oven to kill spores of this bacterium. A typical dry air oven sterilization regime would be two hours at 160° C., but other regimes may be applied depending on the microorganism, the culturing medium and others. For dry sterilization, typically fifteen minutes at 121° C. are applied. Tyndallization is the boiling of the culturing medium for ten minutes and cooling. Irradiation may comprise ultraviolet light of 260 nm. It causes the formation of pyrimidine dimers in DNA leading to genetic damage to cells and their ultimate death. X-rays have efficient germicidal properties, but are unpredictable. Gamma-irradiation can penetrate objects with reasonable efficiency. Chemical substances for killing inducer microorganisms may be any chemical substance that is suitable to kill a microorganism, such as phenol and its derivatives, alcohols such as methanol, ethanol or isopropanol, halides such as chlorine or iodine, aldehydes such as glutaraldehyde and formaldehyde, quaternary ammonium compounds such as cetrimide or benzalkonium chloride, chloroform, ethylene oxide, heavy metal ions such as copper, zinc, mercury or arsenic and dyes such as acridine dyes or ethidium bromide. Methods to do this are known to those skilled in the art and include filtration, centrifugation and washing methods. The preferred method for killing microorganisms as comprised by the present invention is by wet heat, preferably at 121° C. for 20 minutes and one bar overpressure. The killing of the cells may be in the culture medium or may be after separation of the cells from the culture medium. Chemical substances may be added to the culture medium at the appropriate concentration to achieve killing of the microorganism cells or may be added to the cells after separation from the culture medium in an appropriate solution. After the cells have been killed, the chemicals have to be separated from the killed microorganism cells in order not to be harmful to the recipient microorganism. The cells are added to the growth medium of the recipient microorganisms for co-cultivation. Therefore, the inducer or donor cells may be cultivated in a shaking flask, 4 ml of culture may be transferred to a vial in a 24 well plate, the plate may be centrifuged, the supernatant may be transferred to a new plate and both plates may be sterilized and freeze dried. Then 4 mf of fresh medium and the preculture of the recipient may be added, the plate may be incubated for 1 to 7 days and extracts may be prepared.

Moreover, also useful as an inducer for the purposes of the present invention is the medium, in which the inducer microorganism had been cultured and which has been rendered inactive. This can be a solid or aqueous medium, whereby aqueous medium is preferred. After cultivation as referred to above, the inducer microorganism is separated from the medium by any methods known in the art. The remaining culture medium is thereafter inactivated by means known in the art including heat as the preferred inactivation method as referred to above. Preferably, the medium is inactivated by wet heat, more preferably at 121° C. for 20 minutes and one bar overpressure.

The microbial inducers may be pre-cultivated in the same volume of medium as it used later for the induction of the recipient microorganism. By this way, inducers may be excreted into the medium or may be secreted by dead microorganisms or may be released from disintegrated microorganisms. The preincubated microbial cells and/or debris may be removed afterwards to leave the culture medium in which the inducer microorganism had been cultured.

The inactivated culture medium may be used as the new culture medium, to which nutrients may be added in order to allow growth of the recipient microorganism. The nutrients are as mentioned above. Alternatively, the inactivated culture medium may be added to a new culturing medium comprising any substances suitable to allow the growth of the recipient microorganism. The skilled person is thereby capable of adapting the new culture medium and the inactivated medium to allow growth of the recipient microorganism and the activation of silent genes therein, as, e.g., expressed by an inhibitory activity of the inactivated medium.

In another embodiment, the solutes in the medium are concentrated and inactivated. Concentration of the medium by removing the solvent can be performed by any method known in the art, including evaporation, vacuum concentration, lyophilization, reverse extraction, solute precipitation and dialysis (solvent exchange). The objective of solvent removal is to preserve solutes and to concentrate the solutes. The preferred concentration method comprised by the present invention is lyophilization. Thereby, there is no restriction with respect to the sequence of applying the concentration or inactivation step. In one embodiment, the solutes may first be concentrated and thereafter inactivated or the solutes may first be inactivated and then concentrated.

In evaporation, two approaches can be used for solvent removal, one being by boiling (by applying heat or a vacuum) and the other by directing a stream of (inert) gas over the solvent. In this latter approach, the gas essentially extracts solvent from the liquid phase by dissolving it into a gaseous stream. This is the basis of gas chromatography. In vacuum concentration devices, a vacuum pump is attached to an airtight, low speed centrifuge that, when running, prevents bumping by forcing the liquid down into the tube. The system can then run at high vacuum levels to speed solvent removal. Similar to vacuum concentration, the process of lyophilization goes one step further by lowering sample temperature to the point where the solution freezes and solvents are removed by sublimation. The freezing step can be done in the same preparation step or caused by the application of a vacuum which, in the process of removing the atmosphere, also removes heat. Normally the solution is always frozen before the vacuum is applied. Reverse extraction can also be used for solvent removal. Reverse extraction works in the same way as extraction, except that the options not selected are extracted instead of extracting the options that are selected. For example, small volumes of solutes in aqueous buffer are concentrated by adding dry n-butanol. Water is miscible with the alcohol while the solutes are not, resulting in a net flow of water into the butanol phase, which results in a higher concentration of solutes in the remaining (original) aqueous buffer. In dialysis, semi-permeable membranes are used for removing small solutes and solvents from solutions. Centrifugal concentration through a semi-permeable membrane and dialyzing solvents by mass action are further dialysis methods for concentrating solutes by dialysis. In both cases, membranes with controlled pore size allow low molecular weight solutes and solvents to pass through the membrane while retaining the larger molecules. Centrifugal concentrators use centrifugal force to push the solution through the membrane while dialysis utilizes diffusion. Solvents can be removed by dialysis against concentrated solutions containing large molecular weight compounds or against a substance in the solid phase miscible in the dialyzed solvent. Precipitation is the condensation of a solid from a solution during a chemical reaction. Precipitation may occur if the product of the reaction is insoluble in the reaction solvent. Thus, it precipitates as it is formed. The precipitate may easily be separated by filtration, decanting, or centrifugation.

In a preferred embodiment of the present invention, the inducer microorganisms are separated from the medium by centrifugation, the supernatant is concentrated by lyophilization, the lyophilized product is reconstituted in a medium for use in co-cultivation and the medium with the reconstituted lyophilized product is inactivated by heat, preferably at 121° C. for 20 minutes and one bar overpressure.

Alternatively, the inducer microorganism and the medium in which the inducer microorganism had been cultured are not separated, but are commonly inactivated by any methods suitable to kill the microorganism and inactivate the culture medium. Alternatively, the microorganism is killed and the medium is inactivated separately and used in combination as a microorganism inducer.

In a further embodiment, the inducer may be one or more than one inducer, e.g., more than one chemical inducer, e.g., two or three chemical inducers, or a chemical inducer may be combined with a microorganism inducer, or a killed microorganism inducer may be combined with an inactivated culture medium of an microorganism. Any possible combinations are included herein, as long as silent genes are activated in a recipient microorganism.

The recipient microorganisms are co-cultivated together with the inducer in a co-cultivation medium under the conditions, as referred to above. Consequently, as referred to herein, the co-cultivation medium is a medium allowing the growth of a recipient microorganism under non-induction conditions and comprises an inducer as specified herein.

In a fifth aspect of the present invention, the recipient microorganism is selected from actinobacteria, myxobacteria, bacilli, or fungi.

Actinobacteria are a group of Gram-positive bacteria with high guanine and cytosine content. They can be terrestrial or aquatic. Actinobacteria is one of the dominant phyla of the bacteria. Actinobacteria include some of the most common soil life, freshwater life, and marine life bacteria, playing an important role in decomposition of organic materials, such as cellulose and chitin, and thereby playing a vital part in organic matter turnover and carbon cycle. This replenishes the supply of nutrients in the soil and is an important part of humus formation. Other Actinobacteria inhabit plants and animals, including a few pathogens, such as Mycobacterium, Corynebacterium, Nocardia, Rhodococcus and a few species of Streptomyces. Actinobacteria are well known as secondary metabolite producers and hence of high pharmacological and commercial interest. One example of an antibiotic is actinomycin, however, hundreds of naturally occurring antibiotics have been discovered in these terrestrial microorganisms, especially from the genus Streptomyces. Most actinobacteria of medical or economic significance are in subclass Actinobacteridae, order Actinomycetales. While many of these cause disease in humans, Streptomyces is notable as a source of antibiotics.

Myxobacteria (“slime bacteria”) are a group of bacteria that predominantly live in the soil. Myxobacteria have very large genomes, relative to other bacteria, e.g., 9-10 million nucleotides. Myxobacteria are included among the delta group of proteobacteria, a large taxon of Gram-negative forms. Myxobacteria can move actively by gliding. They typically travel in swarms, containing many cells kept together by intercellular molecular signals. This close concentration of cells may be necessary to provide a high concentration of extracellular enzymes used to digest food. Myxobacteria produce a number of biomedically and industrially useful chemicals, such as antibiotics, and export those chemicals outside of the cell. Metabolites secreted by Sorangium cellulosum, known as epothilones, have been noted to have antineoplastic activity. This has led to the development of analogs that mimic its activity. One such analog, known as Ixabepilone, is an approved chemotherapy agent for the treatment of metastatic breast cancer.

Bacillus is a genus of Gram-positive, rod-shaped bacteria. Bacillus species can be obligate aerobes or facultative anaerobes. Ubiquitous in nature, Bacillus includes both free-living and pathogenic species. Under stressful environmental conditions, the cells produce oval endospores that can stay dormant for extended periods. These characteristics originally defined the genus, but not all such species are closely related, and many have been moved to other genera. Many Bacillus species are able to secrete large quantities of enzymes. Bacillus amyloliquefaciens is the source of a natural antibiotic protein barnase (a ribonuclease), alpha amylase used in starch hydrolysis, the protease subtilisin used with detergents, and the BamH1 restriction enzyme used in DNA research.

A fungus is a member of a large group of eukaryotic organisms that includes microorganisms, such as yeasts and moulds. These organisms are classified as a kingdom, Fungi, which is separate from plants, animals, and bacteria. One major difference is that fungal cells have cell walls that contain chitin, unlike the cell walls of plants, which contain cellulose. These and other differences show that the fungi form a single group of related organisms, named the Eumycota (true fungi or Eumycetes). Many species produce metabolites that are major sources of pharmacologically active drugs. Particularly important are the antibiotics, including the penicillins, a structurally related group of β-lactam antibiotics that are synthesized from small peptides. Although naturally occurring penicillins, such as penicillin G (produced by Penicillium chrysogenum), have a relatively narrow spectrum of biological activity, a wide range of other penicillins can be produced by chemical modification of the natural penicillins. Modern penicillins are semisynthetic compounds, obtained initially from fermentation cultures, but then structurally altered for specific desirable properties. Other antibiotics produced by fungi include cyclosporin, commonly used as an immunosuppressant during transplant surgery, and fusidic acid, used to help control infection from methicillin-resistant Staphylococcus aureus bacteria. There is widespread use of these antibiotics for the treatment of bacterial diseases, such as tuberculosis, syphilis, leprosy. In nature, antibiotics of fungal or bacterial origin appear to play a dual role: at high concentrations they act as chemical defense against competition with other microorganisms in species-rich environments, such as the rhizosphere, and at low concentrations as quorum-sensing molecules for intra- or interspecies signaling. Other drugs produced by fungi include griseofulvin isolated from Penicillium griseofulvum, used to treat fungal infections, and statins (HMG-CoA reductase inhibitors), used to inhibit cholesterol synthesis. Examples of statins found in fungi include mevastatin from Penicillium citrinum and lovastatin from Aspergillus terreus.

In a sixth aspect of the present invention, the chemical inducer is selected from an anorganic salt of arsenic, plumb, cadmium, cobalt, selenium, nickel, strontium and nitride and/or DMSO. Preferred embodiments of such salts are AsI3, Pb(NO3)2, CdCl2, CoCl2, NaN3, NaHSeO3, NiCl2, and/or SrCl2. More preferably, the salts, such as those as specified, are present in concentrations of 1 to 5 μg/ml of each salt, most preferably the concentrations are 1.6 μg/ml AsI3, 3.3 μg/ml AsI3, 1.6 μg/ml Pb(NO3)2, 3.3 μg/ml Pb(NO3)2, 1.6 μg/ml CdCl2, 3.3 μg/ml CdCl2, 1.6 μg/ml CoCl2, 3.3 μg/ml CoCl2, 1.6 μg/ml NaN3, 3.3 μg/ml NaN3, 1.6 μg/ml NaHSeO3, 3.3 μg/ml NaHSeO3, 1.6 μg/ml NiCl2, 3.3 μg/ml NiCl2, and/or 1.6 μg/ml SrCl2, 3.3 μg/ml SrCl2. DMSO is preferably present in a concentration of 1 to 100 μl/ml, more preferably 10 to 50 μl/ml, most preferably 10 μl/ml DMSO, 30 μl/ml DMSO, or 50 μl/ml DMSO.

In a seventh aspect of the present invention, the microorganism inducer is a pathogenic microorganism or a soil microorganism. A pathogenic or infectious microorganism in general includes a microorganism, such as a virus, bacterium, prion, or fungus that cause disease in its animal or plant host. Soil contamination has the longest or most persistent potential for harbouring a pathogenic microorganism. A soil microorganism is a microorganism present in the soil. There are thousands of different species of bacteria and hundreds of different species of fungi and protozoa in the soil that form the soil microorganisms. All these microorganisms are comprised for the purposes of the present invention. Preferred pathogenic or soil microorganisms as comprised by the present invention are pathogenic or soil bacteria, more preferably selected from the genus Acetobacter, Actinobacillus, Actinomadura, Actinomyces, Actinoplanes, Aeromonas, Alcaligenes, Alteromonas, Amycolatopsis, Arthrobacter, Aureobacterium, Bacillus, Bacteroides, Bifidobacterium, Borella, Brevibacterium, Burkholderia, Campylobacter, Cellulomonas, Clavibacter, Clostridium, Corynebacterium, Enterobacter, Enterococcus, Escherichia, Eubacterium, Flavobacterium, Fusobacterium, Haemophilus, Helicobacter, Klebsiella, Lactobacillus, Legionella, Microbacterium, Micrococcus, Micromonospora, Moraxella, Mycobacterium, Mycoplasma, Myxococcus, Neisseria, Nocardia, Pasteurella, Photorhabdus, Polyangium, Propionibacterium, Preoteus, Pseudomonas, Rhodococcus, Salmonella, Selenomonas, Serratia, Shigella, Sphingomonas, Staphylococcus, Streptococcus, Streptomyces, Thermoactinomyces, Treponema, Tsukamurella, Vibrio, Xanthomonas, Xenorhabdus or Yersinia, or pathogenic or soil fungi, more preferably of the Ascomycota, Basidiomycota, Oomycota, Zygomycota or yeasts. Still more preferred are pathogenic or soil microorganisms of the genus Escherichia, Staphylococcus, Pseudomonas or Candida, still more preferred of the species Escherichia coli, Staphylococcus aureus, Pseudomonas aeruginosa or Candida albicans and most preferred are Escherichia coli ATCC 35218, Staphylococcus aureus ATCC 33592, Pseudomonas aeruginosa ATCC 27853 or Candida albicans ATCC 753.

Candida albicans (C. albicans) is a diploid fungus that grows both as yeast and filamentous cells and a causal agent of opportunistic oral and genital infections in humans. C. albicans is commensal and a constituent of the normal gut flora, comprising microorganisms that live in the human mouth and gastrointestinal tract. C. albicans lives in 80% of the human population without causing harmful effects.

Escherichia coli (E. coli) is a Gram-negative, facultative anaerobic and non-sporulating rod-shaped bacterium. It can live on a wide variety of substrates. E. coli uses mixed-acid fermentation in anaerobic conditions, producing lactate, succinate, ethanol, acetate, and carbon dioxide. Optimal growth of E. coli occurs at 37° C., but some laboratory strains can multiply at temperatures of up to 49° C. Growth can be driven by aerobic or anaerobic respiration, using a large variety of redox pairs, including the oxidation of pyruvic acid, formic acid, hydrogen and amino acids, and the reduction of substrates, such as oxygen, nitrate, dimethyl sulfoxide and trimethylamine N-oxide. E. coli is one of the most explored microorganisms, which is caused by several facts. The first one is a sufficiently easy growth of this bacterium on all basic carbon sources (both at aerobic and anaerobic conditions). The second one is that the E. coli genome has been sequenced completely. Furthermore, it is considered that metabolic functions are observed for more then 80% of genes. The third one is that E. coli cells are very often used in bioengineering studies and biotechnological production.

Staphylococcus aureus (S. aureus) is a facultative anaerobic Gram-positive coccal bacterium. It is frequently found as part of the normal skin flora on the skin and nasal passages. It is estimated that 20% of the human population are long-term carriers of S. aureus. S. aureus is the most common species of staphylococci to cause Staphylococcus infections.

Pseudomonas aeruginosa (P. aeruginosa) is a Gram-negative, aerobic, rod-shaped bacterium with unipolar motility. An opportunistic human pathogen, P. aeruginosa is also an opportunistic pathogen of plants. Its optimum temperature for growth is 37° C., and it is able to grow at temperatures as high as 42° C. The bacterium is ubiquitous in soil and water. Regulation of gene expression can occur through cell-cell communication or quorum sensing (QS) via the production of small molecules called autoinducers. QS is known to control expression of a number of virulence factors. Another form of gene regulation that allows the bacteria to rapidly adapt to surrounding changes is through environmental signaling. Recent studies have discovered anaerobiosis can significantly impact the major regulatory circuit of QS. This important link between QS and anaerobiosis has a significant impact on production of virulence factors of this organism.

In an eighth aspect of the present invention, the method is a high-through-put screening method. High-throughput screening (HTS) is a method for scientific experimentation especially used in drug discovery and relevant to the fields of biology and chemistry. Using for example robotics, data processing and control software, liquid handling devices, and sensitive detectors, high-throughput screening allows a researcher to quickly conduct thousands or even millions of biochemical, genetic or pharmacological tests. Through this process one can rapidly identify active compounds, antibodies or genes which modulate a particular biomolecular pathway. Usually, HTS uses automation to run a screen of an assay against a library of candidate compounds such as a library of LMW compounds. Typical HTS screening libraries or “decks” can contain from 100,000 to more than 2,000,000 compounds.

Most often, the key testing vessel of HTS is the multi-well plate or microplate. Modern microplates for HTS generally have either 96, 384, 1536, or 3456 wells. These are all multiples of 96, reflecting the original 96 well microplate with 8×12 9 mm spaced wells. Most of the wells contain experimentally useful matter, often an aqueous solution of dimethyl sulfoxide (DMSO) and some other chemical compound, the latter of which is different for each well across the plate. The other wells may be empty, intended for use as optional experimental controls.

To prepare for an assay, the researcher fills each well of the plate with some biological entity that he or she wishes to conduct the experiment upon. In the present case the test system comprising a microorganism and an inducer is to be filled in. After some incubation time has passed to allow the inducer to react (or fail to react) with the microorganism in the wells, measurements are taken across all the plate's wells, either manually or by a machine. A specialized automated analysis machine can run a number of experiments on the wells (such as shining polarized light on them and measuring reflectivity, which can be an indication of the growth of the microorganism). In this case, the machine may output the result of each experiment as a grid of numeric values, with each number mapping to the value obtained from a single well. A high-capacity analysis machine can measure dozens of plates in the space of a few minutes like this, generating thousands of experimental data points very quickly.

In a ninth aspect of the present invention, the methods as referred to above are useful for the discovery of a medicament.

In a tenth aspect of the invention, the medicament is an antibiotic.

The methods as described above, which relate to the activation of silent genes, the screening of an inducer or the screening of a recipient microorganism are useful for the detection of a medicament, preferably an antibiotic. In an embodiment, co-cultivation of a recipient microorganism and a killed inducer microorganism or inactivated supernatant of a medium, in which the microorganism had been cultivated, are useful for such purposes. Activation of silent genes by microorganism inducers may allow the identification of compounds on the surface of a killed microorganism or in the inactivated supernatant that are responsible for the activation of silent genes. Such compounds may be candidate compounds for the development of medicaments. As far as such compounds inhibit the growth of the recipient microorganism, such compounds may be candidate compounds for the development of antibiotics. Moreover, the mechanical contact between a recipient microorganism and a killed microorganism inducer may result in the activation of silent genes, e.g., by the activation of a signaling cascade of a metabolic pathway resulting in the activation of a promoter resulting in change of a phenotype, such as inhibition of growth. Such killed microorganism or the compounds involved in the contact between the microorganisms may be useful for the development of medicaments. Also, chemical inducers may be developed into medicaments, in particular antibiotics. Moreover, compounds produced by a recipient microorganism in response to an inducer may be candidate compounds for the development of medicaments. The detection of the effect of an inducer as defined herein on a recipient microorganism may be performed in an indirect way in that the cells or supernatant of an induced recipient microorganism or an extract thereof is cultivated with an assay strain and the change of phenotype in the assay strain is determined. The change of a phenotype of the assay strain is effected by one or more ingredients comprised by the cell, supernatant or extract thereof. The supernatant or extract, or a compound within the supernatant or extract or cell that effects the change of phenotype, may be developed further to a medicament.

For the production of the medicament the identified target or its pharmaceutically acceptable salt has to be in a pharmaceutical dosage form in general consisting of a mixture of ingredients such as pharmaceutically acceptable carriers or auxiliary substances combined to provide desirable characteristics.

The formulation comprises at least one suitable pharmaceutically acceptable carrier or auxiliary substance. Examples of such substances are demineralised water, isotonic saline, Ringer's solution, buffers, organic or inorganic acids and bases as well as their salts, sodium chloride, sodium hydrogencarbonate, sodium citrate or dicalcium phosphate, glycols, such a propylene glycol, esters such as ethyl oleate and ethyl laurate, sugars such as glucose, sucrose and lactose, starches such as corn starch and potato starch, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils such as groundnut oil, cottonseed oil, corn oil, soybean oil, caster oil, synthetic fatty acid esters such as ethyl oleate, isopropyl myristate, polymeric adjuvans such as gelatin, dextran, cellulose and its derivatives, albumins, organic solvents, complexing agents such as citrates and urea, stabilizers, such as protease or nuclease inhibitors, preferably aprotinin, aminocaproic acid or pepstatin A, preservatives such as benzyl alcohol, oxidation inhibitors such as sodium sulphite, waxes and stabilizers such as EDTA. Colouring agents, releasing agents, coating agents, sweetening, flavouring and perfuming agents, preservatives and antioxidants can also be present in the composition. The physiological buffer solution preferably has a pH of approx. 6.0-8.0, especially a pH of approx. 6.8-7.8, in particular a pH of approx. 7.4, and/or an osmolarity of approx. 200-400 milliosmol/liter, preferably of approx. 290-310 milliosmol/liter. The pH of the medicament is in general adjusted using a suitable organic or inorganic buffer, such as, for example, preferably using a phosphate buffer, tris buffer (tris(hydroxymethyl)amino [symbol] methane), HEPES buffer ([4 (2 hydroxyethyl)piperazino]ethanesulphonic acid) or MOPS buffer (3 morpholino-1 propanesulphonic acid). The choice of the respective buffer in general depends on the desired buffer molarity. Phosphate buffer is suitable, for example, for injection and infusion solutions. Methods for formulating a medicament as well as a suitable pharmaceutically acceptable carrier or auxiliary substance are well known to the one of skill in the art. Pharmaceutically acceptable carriers and auxiliary substances are chosen according to the prevailing dosage form and identified compound.

The medicament can be manufactured for oral, nasal, rectal, parenteral, vaginal, topic or vaginal administration. Parental administration includes subcutaneous, intracutaneous, intramuscular, intravenous or intraperitoneal administration.

The medicament can be formulated as various dosage forms including solid dosage forms for oral administration such as capsules, tablets, pills, powders and granules, liquid dosage forms for oral administration such as pharmaceutically acceptable emulsions, microemulsions, solutions, suspensions, syrups and elixirs, injectable preparations, for example, sterile injectable aqueous or oleaginous suspensions, compositions for rectal or vaginal administration, preferably suppositories, and dosage forms for topical or transdermal administration such as ointments, pastes, creams, lotions, gels, powders, solutions, sprays, inhalants or patches.

The specific therapeutically effective dose level for any particular patient will depend upon a variety of factors including the activity of the identified compound, the dosage form, the age, body weight and sex of the patient, the duration of the treatment and like factors well known in the medical arts.

The total daily dose of the compounds identified by the methods of the present invention administered to a human or other mammal in single or in divided doses can be in amounts, for example, from about 0.01 to about 50 mg/kg body weight or more, preferably from about 0.1 to about 25 mg/kg body weight. Single dose compositions may contain such amounts or sub-multiples thereof to make up the daily dose. In general, treatment regimens according to the present invention comprise administration to a patient in need of such treatment from about 10 mg to about 1000 mg of the compound(s) of the compounds of the present invention per day in single or multiple doses.

In an eleventh aspect, the present invention relates to a medium for cultivation of a recipient microorganism comprising an inducer that activates silent genes in the recipient microorganism, wherein the inducer is selected from a chemical inducer and/or a microorganism inducer that is selected from killed microorganism cells and/or inactivated culture medium in which the microorganism cells had been cultured.

Further aspects of the invention define the medium with respect to the activation of silent genes, the recipient microorganism and the inducer. In this respect, reference is made to the definitions as they are given above with respect to the methods of the present invention.

### EXAMPLES

### Example 1

### Preparation of 24-Well Plates with Microbial Inducers (Plates 1 and 2)

The microbial inducer strains Staphylococcus aureus ATCC 33592, Escherichia coli ATCC 35218, and Pseudomonas aeruginosa ATCC 27853 were inoculated in sterile 300 ml Erlenmeyer flasks filled with 100 ml sterile Müller Hinton medium (30% beef infusion; 1.75% casein hydrolysate; 0.15 starch; pH adjusted to neutral at 25 degree Celsius; percentage amounts as w/w). The microbial inducer strain Candida albicans ATCC 753 was inoculated in a sterile 300 ml Erlenmeyer flask filled with 100 ml sterile 5083 medium. The incubation time was 24 hours at 37° C. and 180 rpm. After the incubation, 4 ml of the microbial inducer cell cultures were pipetted into the respective wells of the 24-deep well plate (plate 2, cells) (see Table 2). Hereupon the filled 24-deep well plate was centrifuged at 3500 rpm for 10 minutes and 4 ml of the supernatant in each well was added in a new 24-deep well plate (plate 1, supernatant) by using the same pipetting scheme as before (see Table 1). The filled 24-deep well plates were covered with air permeable foil and stored at −80° C. On the next day the plates were freeze dried at −80° C. and 0.05 mbar vacuum for at least 48 hours. After freeze drying, the 24-deep well plates were filled with 5294 medium (4 ml in each well) and covered with 24-deep well sandwich covers. In this form the filled 24-deep well plates were autoclaved at 121° C. and one bar overpressure for 20 minutes.

TABLE 1
Plate 1 supernatant

| | 1 | 2 | 3 | 4 | 5 | 6 |
|---|---|---|---|---|---|---|
| A | blank | ATCC 33592 | ATCC 33592 | ATCC 33592 | ATCC 33592 | ATCC 33592 |
| | | S. aureus | S. aureus | S. aureus | S. aureus | S. aureus |
| B | blank | ATCC 35218 | ATCC 35218 | ATCC 35218 | ATCC 35218 | ATCC 35218 |
| | | E. coli | E. coli | E. coli | E. coli | E. coli |
| C | blank | ATCC 27853 | ATCC 27853 | ATCC 27853 | ATCC 27853 | ATCC 27853 |
| | | P. aerug.- | P. aerug.- | P. aerug.- | P. aerug.- | P. aerug.- |
| D | blank | FH2173 | FH2173 | FH2173 | FH2173 | FH2173 |
| | | C. albicans | C. albicans | C. albicans | C. albicans | C. albicans |

TABLE 2
Plate 2 cells

| | 1 | 2 | 3 | 4 | 5 | 6 |
|---|---|---|---|---|---|---|
| A | blank | ATCC 33592 | ATCC 33592 | ATCC 33592 | ATCC 33592 | ATCC 33592 |
| | | S. aureus | S. aureus | S. aureus | S. aureus | S. aureus |
| B | blank | ATCC 35218 | ATCC 35218 | ATCC 35218 | ATCC 35218 | ATCC 35218 |
| | | E. coli | E. coli | E. coli | E. coli | E. coli |
| C | blank | ATCC 27853 | ATCC 27853 | ATCC 27853 | ATCC 27853 | ATCC 27853 |
| | | P. aerug.- | P. aerug.- | P. aerug.- | P. aerug.- | P. aerug.- |
| D | blank | FH2173 | FH2173 | FH2173 | FH2173 | FH2173 |
| | | C. albicans | C. albicans | C. albicans | C. albicans | C. albicans |

### Example 2

### Extract Activities and Selectivities

1. Extracts with >50% Activity Against One of the Assay Strains

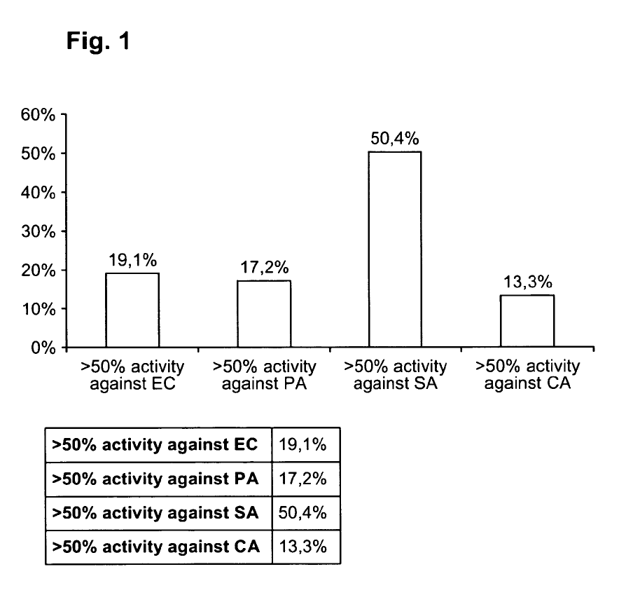

By cultivation of Actinobacteria strains (The Prokaryotes: A Handbook on the Biology of Bacteria (v. 1-7), Martin Dworkin (Editor), Stanley Falkow (Editor), Eugene Rosenberg (Editor), Karl-Heinz Schleifer (Editor), Erko Stackebrandt (Editor), Springer Verlag, 2006; Stackebrandt et al. (1997),) under 32 different cultivation conditions (20 different chemical inducers, inter alia AsI3, Pb(NO3)2, CdCl2, CoCl2, NaN3, NaHSeO3, NiCl2, and/or SrCl2, and/or DMSO; co-incubation of the inducer and the recipient cells in medium 5294), 8 different microbial inducers (Escherichia coli ATCC 35218, Staphylococcus aureus ATCC 33592, Pseudomonas aeruginosa ATCC 27853 and Candida albicans ATCC 753 as cells (co-incubation of inducer cells and recipient cells in medium 5294) and the supernatants thereof (co-incubation of recipient cells in the supernatants)) and 4 different cultivation media (media 5254, 5294, 5567 and 5429), 6912 extracts (polar and nonpolar) were produced. The polar extracts are derived from the supernatants of the cultivation media. The non-polar extracts were produced by freeze-drying of the supernatants, resolving in methanol-water, adsorption to a resin like HP 20 and elution with methanol. Of these 6912 extracts, 3376 showed an additional activity against one or several of the assay strains which were Escherichia coli ATCC 35218, Staphylococcus aureus ATCC 33592, Pseudomonas aeruginosa ATCC 27853 and Candida albicans ATCC 753. The activity of the inducer was detected by the use of the BacTiter-Glo™ assay. By this biological screening, 50.4% of the produced extracts showed >50% activity against the assay strain Staphylococcus aureus ATCC 33592, 19.1% showed >50% activity against the assay strain Escherichia coli ATCC 35218, 17.2% showed >50% activity against the assay strain Pseudomonas aeruginosa ATCC 27853 and 13.3% showed >50% activity against the assay strain Candida albicans ATCC 753 (see FIG. 1).

### 2. Selectivity of the Extracts

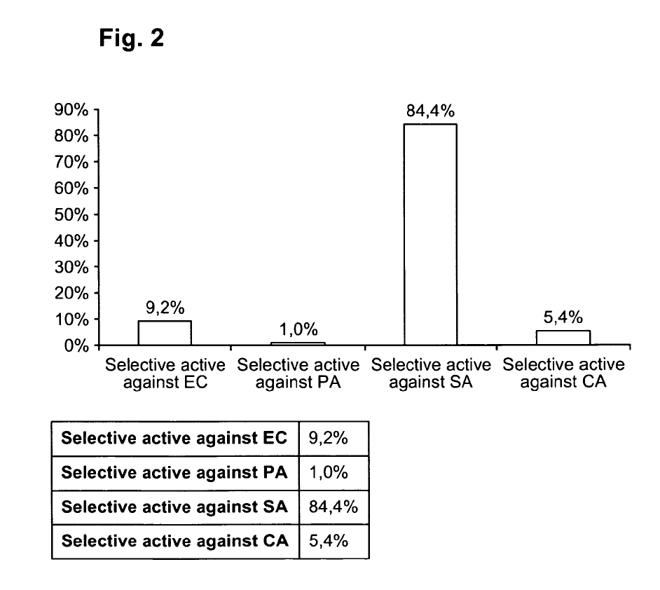

FIG. 2 displays how much of the extracts that showed >50% activity against one of the four assays strains by biological screening (Escherichia coli ATCC 35218, Staphylococcus aureus ATCC 33592, Pseudomonas aeruginosa ATCC 27853 and Candida albicans ATCC 753) were selectively active against these assay strains. (The strains can be purchased from the American Type Culture Collection). 84.4% of the extracts that showed >50% activity against the assay strain Staphylococcus aureus ATCC 33592 were selectively active against this assay strain. 9.2% of the extracts that showed >50% activity against the assay strain Escherichia coli ATCC 35218 were selectively active against this assay strain. 5.4% of the extracts that showed >50% activity against the assay strain Candida albicans ATCC 753 were selectively active against this assay strain and 1.0% of the extracts that showed >50% activity against the assay strain Pseudomonas aeruginosa ATCC 27853 were selectively active against this assay strain (see FIG. 2).

3. Culture Conditions of Extracts that Showed >50% Activity Against Escherichia coli ATCC 35218

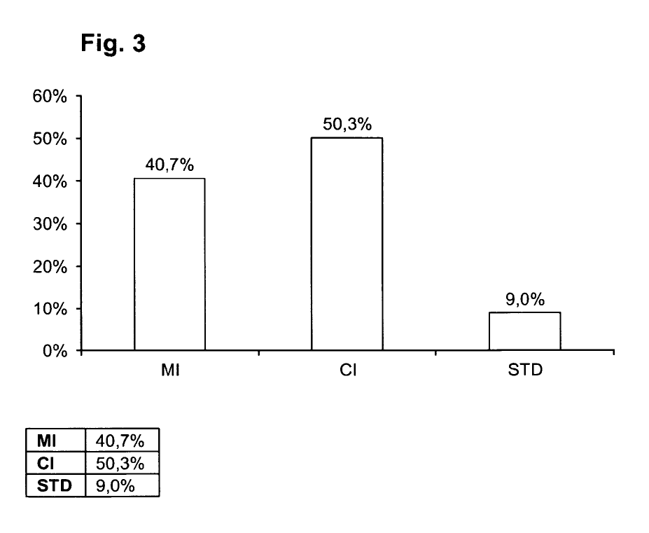

Of the extracts that showed >50% activity against the assay strain Escherichia coli ATCC 35218, 50.3% were produced with cultivation media where different chemical inducers were added. 40.7% were produced by using cultivation media where different microbial inducers were added, and 9.0% with standard cultivation media (see FIG. 3).

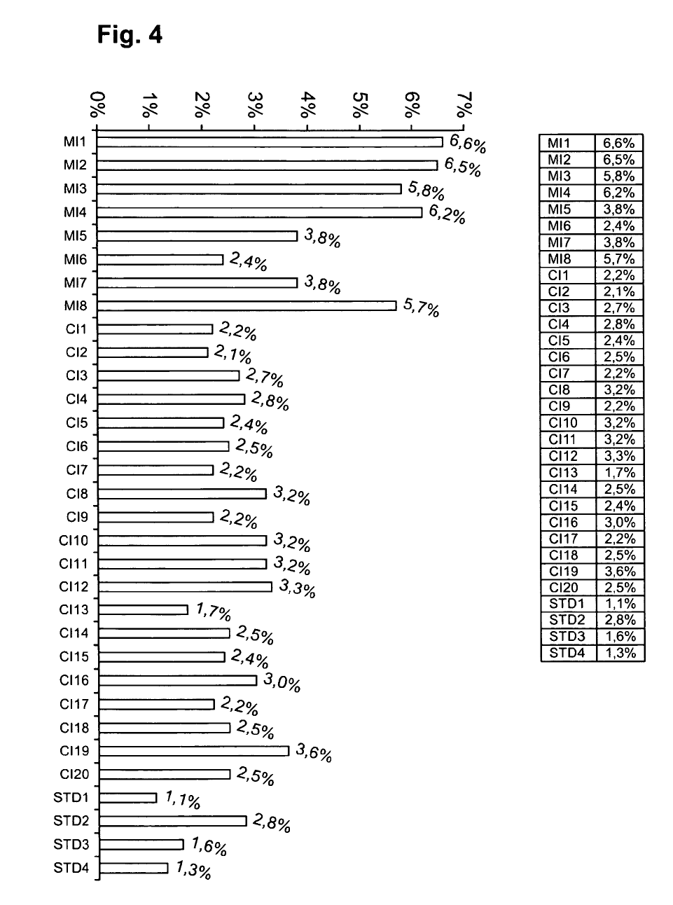

FIG. 4 shows that extracts with an activity against the assay strain Escherichia coli ATCC 35218 higher than 50% were evenly distributed in all media with applications of microbial/chemical inducers and the standard cultivation media, but the microbial inducers had the highest impact. The most effective applied microbial inducers were in this case the supernatant of the Staphylococcus aureus ATCC 33592 cell culture (MI1), with 6.6% extracts that showed >50% activity against the assay strain Escherichia coli ATCC 35218 and the supernatant of the Escherichia coli ATCC 35218 cell culture (MI2), with 6.5% extracts that showed >50% activity against the assay strain Escherichia coli ATCC 35218. The most promising chemical inducers were DMSO (50 μl/ml; CI19) with 3.6% extracts that showed >50% activity against the assay strain Escherichia coli ATCC 35218, and NaHSeO3 (3.3 μg/ml; CI12) with 3.3% extracts that showed >50% activity against the assay strain Escherichia coli ATCC 35218. The most effective standard cultivation medium was 5294 medium (STD2) with 2.8% extracts that showed >50% activity against the assay strain Escherichia coli ATCC 35218 (see FIG. 4).

4. Culture Conditions of Extracts that Showed >50% Activity Against Pseudomonas aeruginosa ATCC 27853

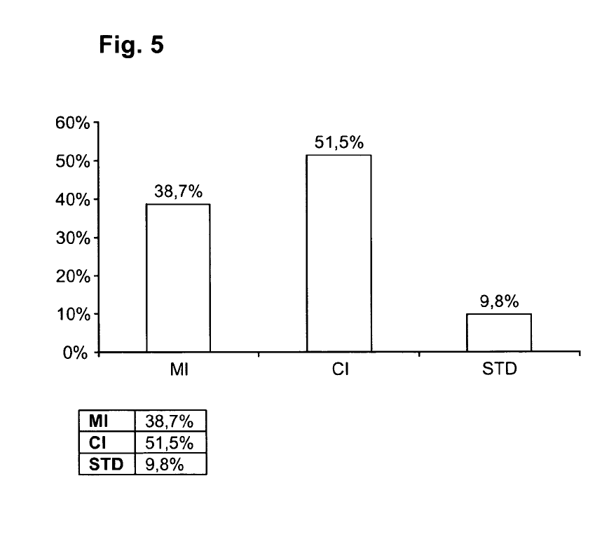

Of the extracts that showed >50% activity against the assay strain Pseudomonas aeruginosa ATCC 27853, 51.5% were produced using cultivation media where different chemical inducers were added. 38.7% of the extracts were produced by using cultivation media where different microbial inducers were added, and 9.8% with standard cultivation media (see FIG. 5).

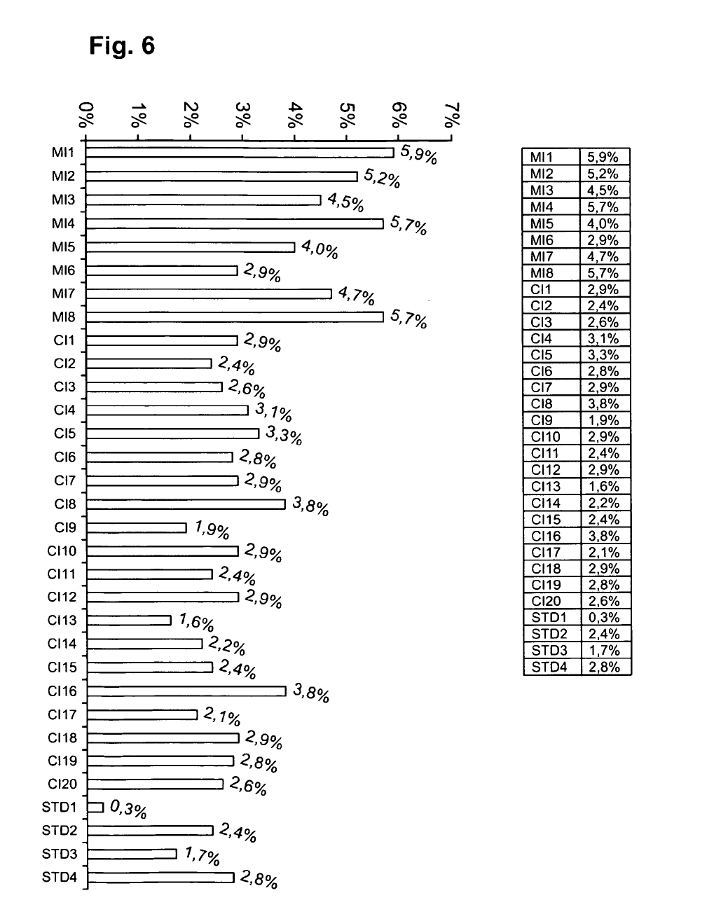

FIG. 6 shows that extracts with an activity against the assay strain Pseudomonas aeruginosa ATCC 27853 higher than 50% are produced more or less equally with application of different microbial/chemical inducers and the standard cultivation media, but the microbial inducers had the highest impact. The most effective microbial inducers were in this case the supernatant of the Staphylococcus aureus ATCC 33592 cell culture (MI1), with 5.9% extracts showing >50% activity against the assay strain Pseudomonas aeruginosa ATCC 27853 and supernatant/cells of the Candida albicans ATCC 753 cell culture (MI4, MI8), each with 5.7% extracts showing >50% activity against the assay strain Pseudomonas aeruginosa ATCC 27853. The most promising chemical inducers were CoCl2 (3.3 μg/ml; CI8) and SrCl2 (3.3 μg/ml; CI16), each with 3.8% extracts showing >50% activity against the assay strain Pseudomonas aeruginosa ATCC 27853. The most effective cultivation medium was 5429 medium (STD4), with 2.8% extracts showing >50% activity against the assay strain Pseudomonas aeruginosa ATCC 27853 (see FIG. 6).

5. Culture Conditions of Extracts that Showed >50% Activity Against Staphylococcus aureus ATCC 33592

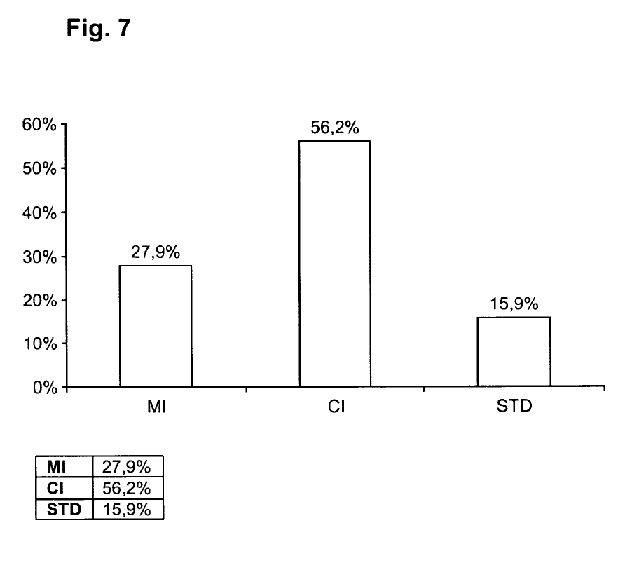

Of the extracts that showed >50% activity against the assay strain Staphylococcus aureus ATCC 33592, 56.2% were produced by using cultivation media where different chemical inducers were added. 27.9% were produced with cultivation media where different microbial inducers were added and 15.9% with standard cultivation media (see FIG. 7).

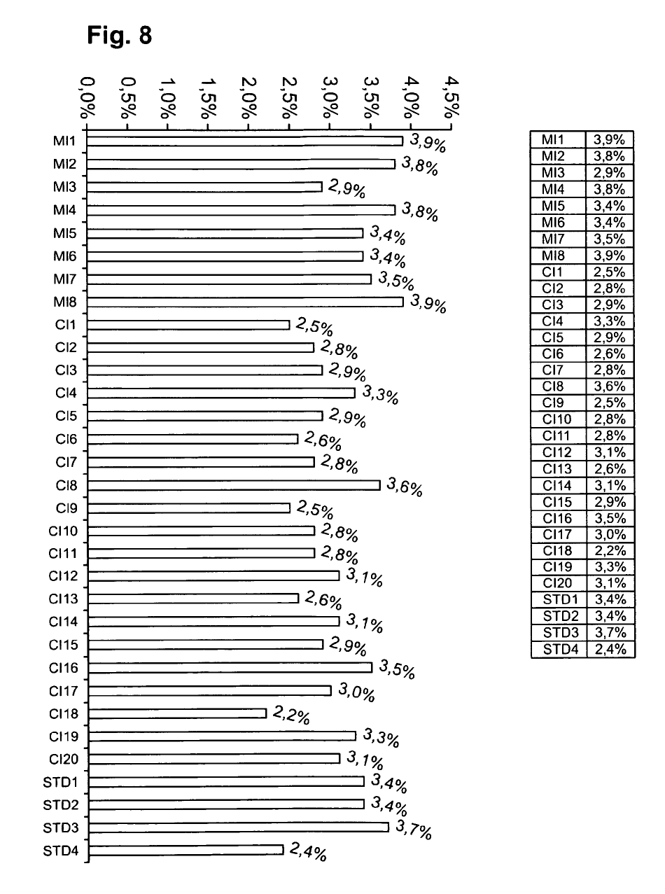

FIG. 8 shows that extracts with an activity against the assay strain Staphylococcus aureus ATCC 33592 higher than 50% are produced more or less equally with application of different microbial/chemical inducer and the standard cultivation media. The most effective microbial inducers were in this case the supernatant of the Staphylococcus aureus ATCC 33592 cell culture (MI1) and the cells of Candida albicans ATCC 753 cell culture (MI8), each with 3.9% extracts that showed >50% activity against the assay strain Staphylococcus aureus ATCC 33592. The most promising chemical inducers were CoCl2 (3.3 μg/ml; CI8) with 3.6% extracts that showed >50% activity against the assay strain Staphylococcus aureus ATCC 33592 and SrCl2 (3.3 μg/ml; CI16) with 3.5% extracts that showed >50% activity against the assay strain Staphylococcus aureus ATCC 33592. The most effective cultivation medium was 5567 medium (STD3), with 3.7% extracts that showed >50% activity against the assay strain Staphylococcus aureus ATCC 33592 (see FIG. 8).

6. Culture Conditions of Extracts that Showed >50% Activity Against Candida albicans ATCC 753

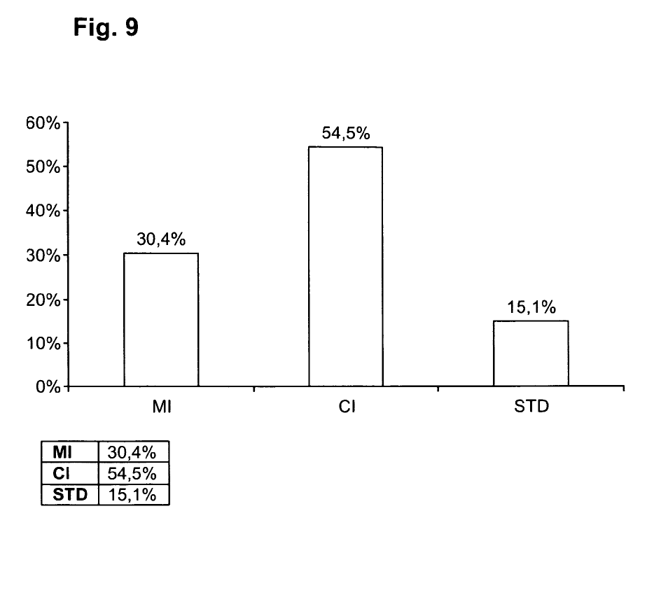

Of the extracts that showed >50% activity against the assay strain Candida albicans ATCC 753, 54.5% were produced by using cultivation media where different chemical inducers were added. 30.4% were produced with cultivation media where different microbial inducers were added and 15.1% with standard cultivation media (see FIG. 9).

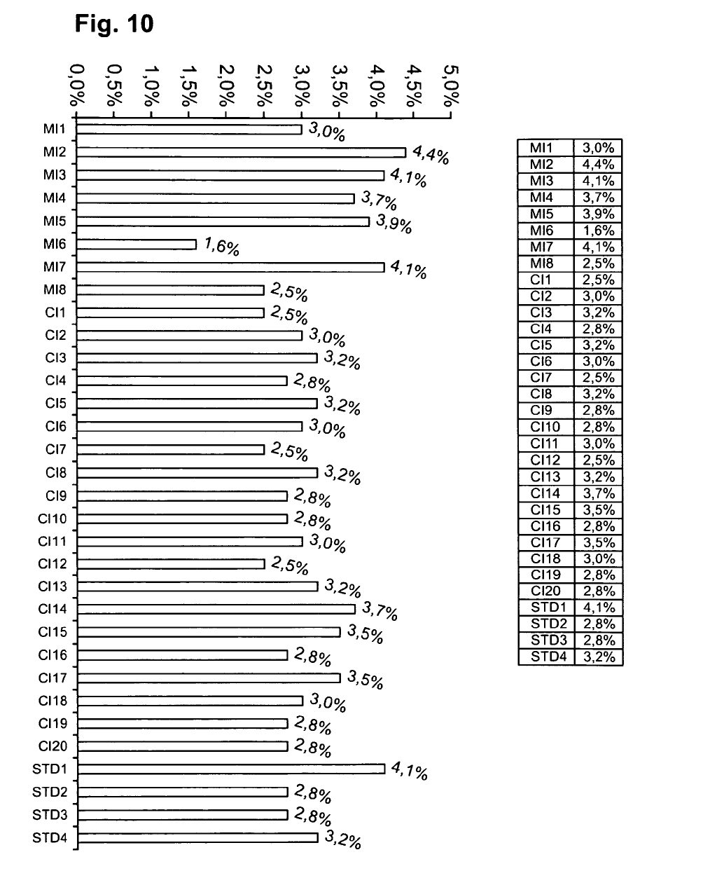

FIG. 10 shows that extracts with an activity against the assay strain Candida albicans ATCC 753 higher than 50% are produced more or less equally with application of different microbial/chemical inducers and the standard cultivation media. The most effective microbial inducers were in this case the supernatant of the Escherichia coli ATCC 35218 cell culture (MI2) with 4.4% extracts that showed >50% activity against the assay strain Candida albicans ATCC 753, the supernatant of Pseudomonas aeruginosa ATCC 27853 cell culture (MI3) and the cells of Pseudomonas aeruginosa ATCC 27853 (MI7), each with 4.1% extracts that showed >50% activity against the assay strain Candida albicans ATCC 753. The most promising chemical inducers were NiCl2 (3.3 μg/ml; CI14), with 3.7% extracts that showed >50% activity against the assay strain Candida albicans ATCC 753 and SrCl2 (1.6 μg/ml; CI15) as well as DMSO (10 μl/ml; CI17) each with 3.5% extracts that showed >50% activity against the assay strain Candida albicans ATCC 753. The most effective standard cultivation medium was 5254 medium (STD1), with 4.1% extracts that showed >50% activity against the assay strain Candida albicans ATCC 753 (see FIG. 10).

### 7. Production of Metabolites by a Recipient Strain Under Co-Incubation Conditions

The recipient strain HAG012128, a strain belonging to the Actinomycetes, was fermented under various induction conditions using different chemical and microbial inductors. The supernatants were obtained. Two active extracts were obtained of which one examined further. In particular, strain HAG012128 was fermented under standard conditions in a standard medium (medium 5294) to which cells of Pseudomonas aeruginosa ATCC 27853 as inducer have been added. A non-polar extract was prepared. The extract was injected at 2 μl in 10-fold concentration on an Agilent 1200 RRLC-system using a 2.6 μm Kinetex RP18 100×2.1 mm column (Phenomenex) and eluted with a gradient of acetonitril/water of 0.6 ml/min 10% to 100% in 15 min. Fractions were collected every 15 seconds to result in 79 fractions. Detection of the 1:1 splitted eluate was recorded by positive ESI-TOF (Agilent G6220A). As a control, HAG012128 was fermented under standard conditions in a standard medium (medium 5294).

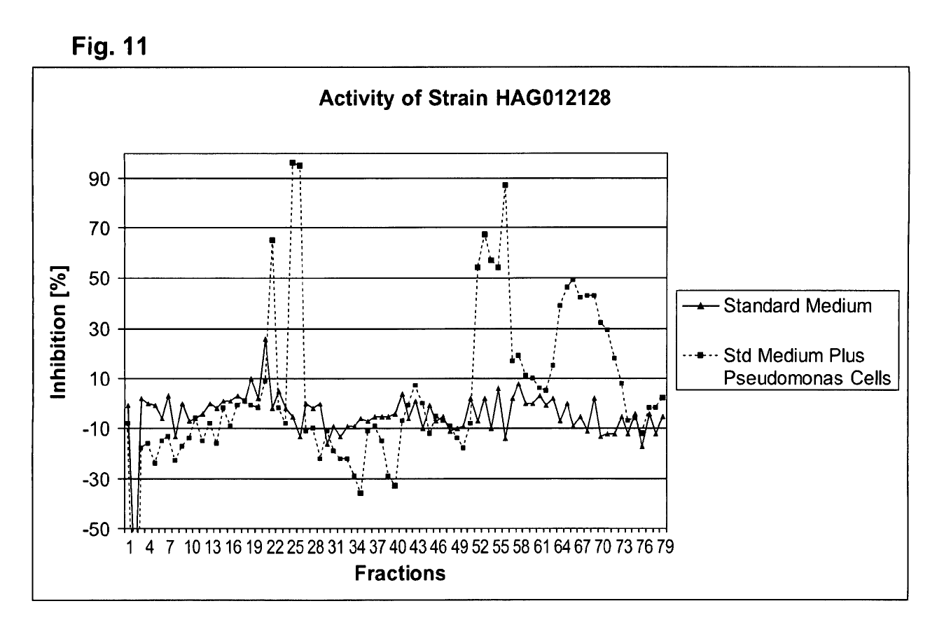

FIG. 11 shows a plot of the inhibition of the assay strain Candida albicans ATCC 753 (y-axis) versus the 79 fractions (x-axis) after HPLC-separation, re-collection and re-testing. Fractions 21 to 23, 51 to 61, and 62 to 74 produce substances that are not produced in the control assay and that inhibit the assay strain vehemently.

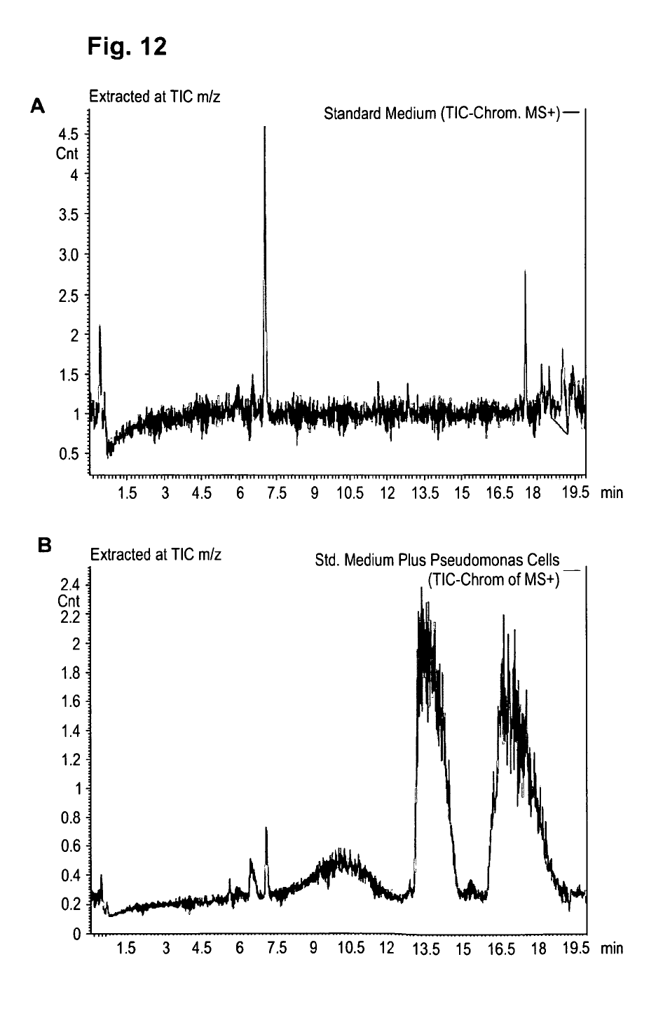

FIG. 12 shows a plot of TIC of positive MS-trace showing the induced Actinomycetes products dinactin (at 13.5 min) and trinactin (at 16.5 min). FIG. 12A shows the control (medium 5294) and FIG. 12B shows the co-incubation experiment. For producing the chromatogram of FIG. 12, the whole extract of the co-incubation assay was used.

The results show that the microorganism inducers have promising effects on the secondary metabolite production (e.g. ATP production) of the analyzed Actinobacteria strains. With focus on the production of extracts that indicate a high activity against the two Gram-negative assay strains, Escherichia coli ATCC 35218 and Pseudomonas aeruginosa ATCC 27853, especially the supernatants of Staphylococcus aureus ATCC 33592, Escherichia coli ATCC 35218, Pseudomonas aeruginosa ATCC 27853 and Candida albicans ATCC 753 cell cultures as well as the cells of Staphylococcus aureus ATCC 33592, Pseudomonas aeruginosa ATCC 27853 and Candida albicans ATCC 753 showed very good results. They produced between 4.0 and 5.9% of the extracts that had >50% activity against the Gram-negative assay strains. The cells of Escherichia coli ATCC 35218 as microorganism inducer exhibited with 2.4 to 2.9% produced extracts with >50% activity against the Gram-negative assay strains a low effect on the production of secondary metabolites with a high activity against the Gram-negative assay strains.

The most effective microorganism inducers, that produce extracts that show a high activity against the Gram-positive assay strain Staphylococcus aureus ATCC 33592, were the supernatant of the Staphylococcus aureus ATCC 33592 cell culture and the cells of Candida albicans ATCC 753 with 3.8 to 3.9% extracts showing >50% activity against this assay strain. The supernatant of Pseudomonas aeruginosa ATCC 27853 showed the lowest activity against the gram-positive assay strain.

With focus on the production of extracts that show a high activity against the assay strain Candida albicans ATCC 753, the best effects were determined by application of the supernatants of Escherichia coli ATCC 35218, Pseudomonas aeruginosa ATCC 27853 and Candida albicans ATCC 753 cell cultures, as well as with the cells of Staphylococcus aureus ATCC 33592 and Candida albicans ATCC 753 as microorganism inducers. They produced 3.7 to 4.4% extracts that showed >50% activity against the assay strain Candida albicans ATCC 753. The lowest effect on the production of secondary metabolites with a high activity against the assay strain Candida albicans ATCC 753 was determined by utilization of Escherichia coli ATCC 35218, and Candida albicans ATCC 753 as microbial inducers. They produced 1.6 to 2.5% extracts showing >50% activity against this assay strain.

Until today, no direct comparable experimental results with regard to the application of Staphylococcus aureus ATCC 33592, Escherichia coli ATCC 35218, Pseudomonas aeruginosa ATCC 27853 and Candida albicans ATCC 753 as microorganism inducers have been published. The above experiments show the usefulness of these microorganisms in killed form or of the inactivated supernatants of media, in which these microorganisms had been cultivated, as inducers which activate silent genes, thereby resulting in growth inhibition of recipient microorganisms.

1. A method for activation of silent genes in a recipient microorganism comprising co-cultivation of a recipient microorganism and an inducer that activates silent genes in the recipient microorganism, wherein the inducer is selected from the group consisting of a chemical inducer, a microorganism inducer, a killed microorganism cell, and inactivated culture medium in which the microorganism cell had been cultured.

2. The method of claim 1 for screening for an inducer that activates silent genes in a recipient microorganism, the method comprising the steps of:
(a) cultivating a recipient microorganism in the presence of a candidate inducer, and
(b) determining the candidate inducer as being an inducer if silent genes are activated in the recipient microorganism,
wherein the candidate inducer is selected from the group consisting of a candidate chemical inducer, a candidate microorganism inducer, a killed microorganism cell, and inactivated culture medium in which the microorganism cell had been cultured.

3. The method of claim 1 for screening for a recipient microorganism, the method comprising the steps of:
(a) cultivating a candidate recipient microorganism in the presence of an inducer that activates silent genes in the recipient microorganism, and
(b) determining the candidate recipient microorganism as being a recipient microorganism if silent genes are activated in the candidate recipient microorganism,
wherein the inducer is selected from the group consisting of a chemical inducer, a microorganism inducer, a killed microorganism cell, and inactivated culture medium in which the microorganism cell had been cultured.

4. The method of claim 1, wherein the activation of silent genes results in a change of a phenotype of the recipient microorganism, wherein the change of phenotype is a change of production of metabolites, a change of growth, and/or a change of morphology.

5. The method of claim 1, wherein the recipient microorganism is a microorganism selected from the group consisting of actinobacteria, myxobacteria, bacilli, and or fungi.

6. The method of claim 1, wherein the chemical inducer is selected from the group consisting of an anorganic salt of arsenic, plumb, cadmium, cobalt, selenium, nickel, strontium and/or nitride.

7. The method of claim 6, wherein the chemical inducer is AsI3, Pb(NO3)2, CdCl2, CoCl2, NaN3, NaHSeO3, NiCl2, and/or SrCl2, or DMSO.

8. The method of claim 1, wherein the microorganism inducer is a pathogenic microorganism or a soil microorganism selected from the group consisting of genus Acetobacter, Actinobacillus, Actinomadura, Actinomyces, Actinoplanes, Aeromonas, Alcaligenes, Alteromonas, Amycolatopsis, Arthrobacter, Aureobacterium, Bacillus, Bacteroides, Bifidobacterium, Borella, Brevibacterium, Burkholderia, Campylobacter, Cellulomonas, Clavibacter, Clostridium, Corynebacterium, Enterobacter, Enterococcus, Escherichia, Eubacterium, Flavobacterium, Fusobacterium, Haemophilus, Helicobacter, Klebsiella, Lactobacillus, Legionella, Microbacterium, Micrococcus, Micromonospora, Moraxella, Mycobacterium, Mycoplasma, Myxococcus, Neisseria, Nocardia, Pasteurella, Photorhabdus, Polyangium, Propionibacterium, Preoteus, Pseudomonas, Rhodococcus, Salmonella, Selenomonas, Serratia, Shigella, Sphingomonas, Staphylococcus, Streptococcus, Streptomyces, Thermoactinomyces, Treponema, Tsukamurella, Vibrio, Xanthomonas, Xenorhabdus or Yersinia.

9. The method of claim 1, wherein the microorganism inducer is a pathogenic or soil fungus selected from the group consisting of Ascomycota, Basidiomycota, Oomycota, Zygomycota, yeasts, Escherichia coli ATCC 35218, Staphylococcus aureus ATCC 33592, Pseudomonas aeruginosa ATCC 27853, and Candida albicans ATCC 753.

10. The method of claim 1, wherein the method is a high-throughput method.

11. A medium for cultivation of a recipient microorganism comprising an inducer that activates silent genes in the recipient microorganism, wherein the inducer is selected from the group consisting of a chemical inducer, a microorganism inducer, a killed microorganism cell, and inactivated culture medium in which the microorganism cell had been cultured.

12. The medium according to claim 11, wherein the activation of silent genes results in a change of a phenotype of the recipient microorganism, wherein the change of phenotype is a change of production of metabolites, a change of growth, and/or a change of morphology.

13. The medium of claim 11, wherein the recipient microorganism is a microorganism selected from the group consisting of actinobacteria, myxobacteria, bacilli, and fungi.

14. The medium of claim 11, wherein the chemical inducer is selected from the group consisting of an anorganic salt of arsenic, plumb, cadmium, cobalt, selenium, nickel, strontium and/or nitride.

15. The medium according to claim 14, wherein the chemical inducer is AsI3, Pb(NO3)2, CdCl2, CoCl2, NaN3, NaHSeO3, NiCl2, and/or SrCl2, or DMSO.

16. The medium of claim 11, wherein the microorganism inducer is a pathogenic microorganism or a soil microorganism selected from the group consisting of genus Acetobacter, Actinobacillus, Actinomadura, Actinomyces, Actinoplanes, Aeromonas, Alcaligenes, Alteromonas, Amycolatopsis, Arthrobacter, Aureobacterium, Bacillus, Bacteroides, Bifidobacterium, Borella, Brevibacterium, Burkholderia, Campylobacter, Cellulomonas, Clavibacter, Clostridium, Corynebacterium, Enterobacter, Enterococcus, Escherichia, Eubacterium, Flavobacterium, Fusobacterium, Haemophilus, Helicobacter, Klebsiella, Lactobacillus, Legionella, Microbacterium, Micrococcus, Micromonospora, Moraxella, Mycobacterium, Mycoplasma, Myxococcus, Neisseria, Nocardia, Pasteurella, Photorhabdus, Polyangium, Propionibacterium, Preoteus, Pseudomonas, Rhodococcus, Salmonella, Selenomonas, Serratia, Shigella, Sphingomonas, Staphylococcus, Streptococcus, Streptomyces, Thermoactinomyces, Treponema, Tsukamurella, Vibrio, Xanthomonas, Xenorhabdus or Yersinia.

17. The medium of claim 11, wherein the microorganism inducer is a pathogenic or soil fungus selected from the group consisting of Ascomycota, Basidiomycota, Oomycota, Zygomycota, yeasts, Escherichia coli ATCC 35218, Staphylococcus aureus ATCC 33592, Pseudomonas aeruginosa ATCC 27853, and Candida albicans ATCC 753.
